(19) United States
(12) Patent Application Publication
(10) Pub. No.: US 2025/0387342 A1
(43) Pub. Date: Dec. 25, 2025

(54) METHODS FOR MANUFACTURING AND USING EXTRACELLULAR VESICLES

(71) Applicant: TheraXyte Bioscience Hong Kong Limited, Hong Kong (CN)

(72) Inventor: Tong ZHAO, Hong Kong (CN)

(21) Appl. No.: 18/880,082

(22) PCT Filed: Jun. 29, 2023
(86) PCT No.: PCT/CN2023/104316

§ 371 Date: Dec. 30, 2024

(30) Foreign Application Priority Data
Jun. 29, 2022 (WO) PCT/CN2022/102338

(51) Int. Cl. A61K 9/50; C07K 14/705; C12N 9/22; C12N 15/113; C12N 15/85

(52) U.S. Cl. CPC A61K 9/5068; A61K 9/5089; C07K 14/70596; C12N 9/222; C12N 15/113; C12N 15/85; C12N 2310/11; C12N 2310/20; C12N 2310/3233

(57) ABSTRACT

Disclosed herein are methods of enhancing extracellular vesicle production.

### CROSS-REFERENCE TO RELATED APPLICATIONS

This application claims priority to Chinese Patent Application No. PCT/CN2022/102338, filed on Jun. 29, 2022, the contents of each of which are hereby incorporated by reference.

### TECHNICAL FIELD

The disclosure relates to methods of manufacturing and using extracellular vesicles. In particular, the disclosure provides methods of enhancing extracellular vesicles production in cells and programmable engineering extracellular vesicles.

### BACKGROUND OF THE INVENTION

Extracellular vesicles (EVs) are naturally derived and secreted by all cells of prokaryotes and eukaryotes. Upon secretion in both normal and pathophysiological conditions, the EVs facilitate the intercellular communication process by acting as cargo-ensembles for transporting essential cellular components (i.e., soluble proteins or active enzymes, lipids, and nucleic acids such as mRNAs, micro-RNAs, long non-coding RNAs, and metabolites).

Such transportation capability of EVs prompted the exploration of the use of EVs in delivering an agent (e.g., a therapeutic agent) to or within a target cell. Compared to other well-known synthetic drug delivery vehicles (e.g., liposome, lipid nanoparticles or viral vectors, etc.), EVs provide numerous advantages as drug carriers due to their characteristics of being natural secretomes from cells for short- or long-distance intercellular communication; having tropism for specific organs or cells via binding to certain surface receptors; superior in cargo trafficking efficiency due to their multiple cell uptake routes which may include endocytosis, phagocytosis, micropinocytosis, or direct fusion with the recipient cell membranes; and ability to avoid immunological clearance owing to the intrinsic nature of the carrier.

The EVs are broadly categorized into ectosomes and exosomes. The exosomes typically have an average diameter range of about 40 to about 160 nm, which is smaller than red blood cells. Exosomes are also highly effective in passing through the blood-brain barrier, and such ability makes them even more enticing for their uses in various types of brain disease drug delivery. However, manufacturing of the exosomes in a large-scale production is challenging due to the limited amounts of naturally produced exosomes in cells.

Thus, there is a need in the art for enhanced EVs manufacturing methods, in particular, enhancing the exosome production in mammalian cells. The present disclosure addresses these needs.

### SUMMARY

Disclosed herein are methods of manufacturing extracellular vesicles (EVs). In particular, the present disclosure provides methods of enhancing the exosome manufacturing process.

In one aspect, the disclosure provides a method of enhancing production of the extracellular vesicles (EVs) that comprises the steps of a) genetically engineering a producer cell to overexpress at least one or more polypeptides and b) harvesting a plurality of EVs from the producer cells. In some cases, the polypeptide is linked to a glycosyl-phosphatidyl-inositol (GPI) group. In some cases, the polypeptide is derived from any one of polypeptides in Table A. In some cases, the polypeptide is derived from a protein selected from the group consisting of CD52, CD55, CD58, CD59, CD109, GPC1, GPC4, and GPC6. In some cases, the polypeptide is derived from CD59. In some cases, polypeptide is derived from CD55. In some cases, the polypeptide is selected from the group consisting of CD52, CD55, CD58, and CD59. In some cases, the polypeptide is CD59. In some cases, polypeptide is CD55. In some cases, the EVs are ectosomes, exosomes, microvesicles, apoptotic bodies, or any combination thereof. In some cases, the EVs are exosomes.

In some cases, the producer cell is genetically engineered by transfecting a recombinant vector system. In some cases, the recombinant vector system comprises a nucleic acid sequence encoding the polypeptide. In some cases, the recombinant vector system comprises an expression control sequence operably linked to the nucleic acid sequence. In some cases, the nucleic acid sequence comprises at least one fluorescent marker. In some cases, the expression control sequence is a promoter. In some cases, the recombinant vector system comprises a selection marker. In some cases, the producer cell is a genetically engineered stable cell line. In some cases, the plurality of EVs is harvested by dialysis or ultra-centrifugation. In some cases, the plurality of EVs is harvested by ultra-centrifugation.

In another aspect, the present disclosure provides a method of making an EV producing stable cell line. In some cases, the method comprises the steps of a) transfecting an EV producer cell with an expression vector, wherein the expression vector comprises a nucleic acid sequence of at least one polypeptide and a selection marker, wherein the polypeptide is linked to a glycosyl-phosphatidyl-inositol (GPI) group; b) screening and selecting the transfected cells; and c) cultivating the selected cells. In some cases, the polypeptide is a protein selected from the group consisting of CD52, CD55, CD58, CD59, CD109, GPC1, GPC4, and GPC6. In some cases, the polypeptide is CD59. In some cases, the polypeptide is CD55. In some cases, the polypeptide is derived from a protein selected from the group consisting of CD52, CD55, CD58, and CD59. In some cases, the polypeptide is derived from CD59. In some cases, the polypeptide is derived from CD55. In some cases, the expression vector comprises an expression control sequence operably linked to the nucleic acid sequence. In some cases, the expression control sequence is a promoter. In some cases, the nucleic acid sequence comprises at least one fluorescent marker. In some cases, the selection marker is selected the group consisting of neomycin resistance, puromycin resistance, hygromycin resistance, DHFR resistance, GPT resistance, zeocin resistance, G418 resistance, phleomycin resistance, blasticidin resistance, and histidinol resistance.

In some cases, the amount of concentration of the harvested EVs from the producer cells is at least 2-fold higher than those from a control cell. In some cases, the amount of concentration of the harvested EVs from the producer cells is 2-fold to 40-fold higher than those from a control cell. In some cases, the producer cell is a mammalian cell. In some cases, the producer cell is a stem cell, mesenchymal stem cell (MSC), HEK 293F cell, HEK 293T cell, or any combination thereof.

In some cases, the EVs are loaded with cargo molecules. In some cases, the cargo molecules comprise an active pharmaceutical ingredient (API). In some cases, the API comprises small molecule therapeutics. In some cases, the cargo molecule comprises a polypeptide, protein, lipid, nucleic acid, carbohydrate, lipid, metabolite, or any combinations thereof. In some cases, the nucleic acid comprises DNA. In some cases, the nucleic acid comprises peptide nucleic acids (PNAs). In some cases, the nucleic acid comprises RNA. In some cases, wherein the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (lncRNA). In some cases, the protein comprises an antibody or enzyme. In some cases, the cargo molecule comprises an antisense oligonucleotide. In some cases, the cargo molecule comprises a morpholino oligomer. In some cases, the cargo molecule comprises one or more components of a gene editing system. In some cases, the gene editing system is selected from the group consisting of CRISPR/Cas, zinc finger nuclease, transcription, and activator-like effector nuclease (TALEN).

In yet another aspect, the present disclosure provides a cell line manufactured according to any one of the methods described herein. The present disclosure also provides a kit for enhancing EV production that comprises any one of the producer cells or the stable cell lines described herein. The present disclosure also provides a composition that comprises a plurality of EVs produced according to any one of the EV production methods described herein. In some cases, the composition further comprises a pharmaceutically acceptable excipient.

In some embodiments, the present disclosure provides a composition comprising an extracellular vesicles (EVs) producer cell, wherein the EV producer cell is genetically engineered to overexpress at least one or more polypeptides, wherein the polypeptide is linked to a glycosyl-phosphatidyl-inositol (GPI) group.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts the protein domain structures of CD55 and artificial CD55. WT CD55 includes a signal peptide domain, a sushi domain, and a GPI anchor (top) and the artificial CD55 includes mCherry fused N- and C-terminal domains of CD55 (bottom) as described in Example 1.

FIG. 1B depicts representative immunoblot images of CD59, CD46, CD55, and GAPDH (loading control) in CD59 (293T_{CD59}), CD46 (293T^{CD46}), and CD55 (293T^{CD55}) overexpressed HEK 293T cells as described in Example 1.

FIG. 2 is a schematic overview of the small extracellular vesicles (sEVs) production process as described in Example 2.

FIG. 3A-B depict the concentration of the harvested extracellular vesicles (EVs) (particles/L) from WT, 293T^{CD46}, 293T^{CD59}, and 293T^{CD55} cells (FIG. 3A) and concentration of the extracellular vesicles (EVs) (ug/mL) from WT, 293T^{CD46}, 293T^{CD59} and 293T^{CD55} cells (FIG. 3B) as described in Example 2.

FIGS. 4A-C depict the characteristics of the harvested extracellular vesicle (EV) including particle size distribution as described in Example 2. FIG. 4A depicts the nFCM scatter plots data of the beads standard. FIG. 4B depicts the nFCM histogram data of the beads standard. FIG. 4C. depicts the size and distribution of the harvested extracellular vesicles (EVs) from WT, 293T^{CD46}, 293T^{CD59}, and 293T^{CD55} cells as described in Example 2.

FIG. 5 depicts the representative immunoblots of Alix, TSG101, PTGFRN, CD59, CD46, CD55, and GAPDH (loading control) from WT, 293T^{CD59}, 293T^{CD46}, and 293T^{CD55} cells as described in Example 2.

FIG. 6 depicts the representative TEM images of the harvested EVs of FIG. 5 as described in Example 2.

FIG. 7 depicts the GPI-anchor proteins identified to be enriched in the EVs harvested from WT, 293T^{WT}, 293T^{CD46}, 293T^{CD59}, and 293T^{CD55} cells as described in Example 2.

FIGS. 8A-C depict representative immunoblot image of CD52 (FIG. 8A); concentration of the extracellular vesicles (EVs) (particles/L) from WT and 293T^{CD52} cells (FIG. 8B); and morphology of the harvested EVs from the 293T^{CD52} cells (FIG. 8C).

FIGS. 9A-C depict representative immunoblot images of CD55 and CD59 (FIG. 9A); concentration of the harvested extracellular vesicles (EVs) (particles/L) from WT, 293T^{CD55}, 293T^{CD59} and 293T^{CD55&CD59} cells (FIG. 9B); and morphology of the harvested EVs from 293TCD55, 293TCD59 and 293TCD55&CD59 cells (FIG. 9C).

FIGS. 10A-C depict representative immunoblot images of CD55 (FIG. 10A); concentration of the harvested extracellular vesicles (EVs) (particles/L) from 293F^{WT} and 293F^{CD55} cells (FIG. 10B); and morphology of the harvested EVs from the 293F^{WT} and 293F^{CD55} cells (FIG. 10C).

FIGS. 11A-C depict representative immunoblot images of CD55 (FIG. 11A); concentration of the harvested extracellular vesicles (EVs) (particles/L) from WT and CD55 overexpressed mesenchymal stem cells (MSC) (FIG. 11B); and morphology of the harvested EVs from the MSC^{WT} and MSC^{CD55} cells (FIG. 11C).

FIG. 12A depicts the HA tagged protein domain structures of CD55 (4) and truncated CD55 (3-0) having a signal peptide domain, a sushi domain, and/or a GPI anchor as described in Example 4.

FIGS. 12B-C depict the expression of the 293F^{WT} and 293F⁴⁻⁰ of FIG. 12A (FIG. 12B) and concentration of the harvested extracellular vesicles (EVs) from the 293F^{WT} and 293F⁴⁻⁰ producer cells as described in Example 4.

FIG. 12D depicts the morphologies of the extracellular vesicles from the HA tagged protein domain structures of CD55 (4) and truncated CD55 (3-0) in Example 4.

FIGS. 13A-D depict the protein domain structures of CD55 (top) and two CD55-based polypeptides (Short and Long) having a signal peptide domain, a sushi domain, a GPI anchor, and or mCherry; the expression of the WT and truncated constructs of FIG. 13A (FIG. 13B); and concentration of the extracellular vesicles (EVs) from the producer cells (FIG. 13C); and cellular uptake of the mCherry-loaded EVs (FIG. 13D) as described in Example 5.

FIGS. 14A-E depicts the protein domain structure of CD46 and two CD46-based polypeptides (Short and Long) having a signal peptide domain, an extracellular domain, a transmembrane domain, a cytoplasmic domain and/or mCherry (FIG. 14A); expression of the WT and the CD46 S and L polypeptides (FIG. 14B); concentration of the extracellular vesicles (EVs) from the producer cells (FIG. 14C); protein domain structures of the CD46-based polypeptides encoding nanoluciferase (Nluc) near the N-terminal (outside) or C-terminal (inside) (FIG. 14D); and immunoblot images of CD46 from the producer cells as described in Example 5 (FIG. 14E).

FIGS. 15A-B depict the concentrations of the extracellular vesicles (EVs) from CD55, CD55/CD59, and CD55/CD46-Short producer cells (FIG. 15A); representative immunoblot images of CD55 and mOrange from the producer cells and concentrations of the EVs from 293F based WT, CD55, CD55/CD63-mOrange, CD55/CD81-mOrange, and CD55/CD9-mOrange producer cells (FIG. 15B) as described in Example 5.

FIGS. 16A-F depict the cell viability and percentage of extracellular vesicles production in 28F and 293F cells cultured for different days (FIG. 16A); the cell status (density and viability) and concentration of extracellular vesicles production in 293F cells grown in different cell medium (FIG. 16B); the cell status (density and viability) and concentration of extracellular vesicles production in 28F cells grown in different cell medium (FIG. 16C); percentage of extracellular vesicles production in 293F cells grown in the presence or absence of an anti-clump agent (FIG. 16D); the cell status (density and viability) and concentration of extracellular vesicles production based on the different cell culture methods (FIG. 16E); and the ratio of permeate and retentate following filtration using the tested filters as described in Example 6 (FIG. 16F).

FIG. 17 depicts the downstream purification process as described in Example 6.

FIGS. 18A-E depict the size distribution and EV percentage of the 28F harvested extracellular vesicles (EVs) (FIG. 18A); TEM images (FIG. 18B); Cryo-EM images (FIG. 18C); nFCM plot (FIG. 18D); and immunoblot of CD55, Alix, TSG101, CD9, CD63, CD81, and CYC1 in cells and EVs (FIG. 18E) as described in Example 6.

FIGS. 19A-B depict the stability of the harvested extracellular vesicles (EVs) over time under different temperatures or following lyophilization as described in Example 6.

FIGS. 20A-B depict the outside (FIG. 20A) or inside (FIG. 20B) EVs loading of the siRNA as described in Example 6.

FIG. 21 depicts the nFCM plots of the siRNA loaded extracellular vesicles (EVs) with or without RNAse treatment as described in Example 6.

FIGS. 22A-C depict the RNA loading efficiencies in extracellular vesicles (EVs) at different concentrations of EVs (FIG. 22A); ratio of the EVs versus RNA (FIG. 22B); and voltages (FIG. 22C) as described in Example 7.

FIG. 23 depicts the nFCM graph showing the HA loading within the lumen (inside) or outside of the extracellular vesicles (EVs) as described in Example 7.

FIGS. 24A-F depict representative immunoblot images (FIG. 24A); response ratio (FIG. 24B) of the cargo molecule BLAM loaded inside or outside of the extracellular vesicles in the presence or absence of Vesicular Stomatitis Virus Glycoprotein (VSVG); representative immunoblots of BLAM and VSVG in cells or EVs with or without VSVG overexpression following the target cell uptake of the EVs loaded with the CD63-(FIGS. 24C-D) or CD46-(FIG. 24E-F) based BLAM fusion proteins as described in Example 8.

FIGS. 25A-B depict the effect of siRNA released from inside (FIG. 25A) or outside (FIG. 25B) of the extracellular vesicles (EVs) in the presence or absence of VSVG as described in Example 8.

### DETAILED DESCRIPTION OF THE INVENTION

### Definitions

The term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11. Unless otherwise indicated, some embodiments herein contemplate numerical ranges. When a numerical range is provided, unless otherwise indicated, the range includes the range endpoints. Unless otherwise indicated, numerical ranges include all values and sub ranges therein as if explicitly written out.

The singular forms “a,” “an,” and “the” are used herein to include plural references unless the context clearly dictates otherwise. Accordingly, unless the contrary is indicated, the numerical parameters set forth in this application are approximations that may vary depending upon the desired properties sought to be obtained by the present invention.

Unless otherwise indicated, open terms, for example “contain,” “containing,” “include,” “including,” and the like mean comprising.

The term “agent”, “active pharmaceutical ingredient (API)”, “therapeutics”, “therapeutic agent”, and “drug” are interchangeably used herein and comprise agents with pharmacological effects inducing a biological or medical response in an animal or human tissue or cell system desired by the researcher, veterinary, general practitioner or other physician, comprising changing biological system at molecular level (e.g., acting as inhibitors, activators, or modulators of proteins), the palliation or the symptoms or the disease or disorder treated; said agents can be chemical compounds, biological molecules with therapeutic activity (e.g., siRNAs, miRNAs, anti-miRNAs, shRNAs, etc., antibodies, antibody fragments recognizing specific epitopes), anti-tumor drugs, or radiotherapy drugs.

The term “cargo molecule” refers to any molecules or compounds that are or to be incorporated, capsulated, fused, or injected into a molecule transferring cargo (e.g., vesicles, exosomes, etc.) and may be chemical or biological molecules with or without therapeutic activity.

The term “extracellular vesicles” shall be understood with the meaning commonly known in the art and refers to vesicles containing membrane-coated cytoplasmic portions that are released from cells in the microenvironment. These vesicles represent a heterogeneous population comprising a plurality of types of vesicles, including “exosomes” and microvesicles, or apoptotic bodies, which can be told apart based on size, antigen composition and secretion modes. The terms “therapeutic delivery vesicle” and “therapeutic cargo” shall be understood to relate to any type of vesicle that is, for instance, obtainable from a cell, for instance a microvesicle (any vesicle shedded from the plasma membrane of a cell), an exosome (any vesicle derived from the endo-lysosomal pathway), an apoptotic body (from apoptotic cells), a microparticle (which may be derived from e.g., platelets), an ectosome (derivable from e.g., neutrophiles and monocytes in serum), prostatosome (obtainable from prostate cancer cells), cardiosomes (derivable from cardiac cells), etc. Furthermore, the terms “cargo molecule delivering vesicle” and “delivery vesicle” shall also be understood to potentially also relate to lipoprotein particles, such as LDL, VLDL, HDL and chylomicrons, as well as liposomes, lipid-like particles, lipidoids, etc. Essentially, the present disclosure may relate to any type of lipid-based structure (vesicular or with any other type of suitable morphology) that can act as a delivery or transport vehicle for cargo molecules.

The term “fusion” or “fusion polypeptide” as used herein refers to a recombinant protein of two or more polypeptides. Fusion proteins can be produced, for example, by a nucleic acid sequence encoding one polypeptide is joined to the nucleic acid encoding another polypeptide or a protein domain such that they constitute a single open-reading frame that can be translated in the cells into a single polypeptide harboring all the intended proteins. The order of arrangement of the polypeptides can vary. Fusion polypeptide can include an epitope tag or a half-life extender. Epitope tags include biotin, FLAG tag, c-myc, hemaglutinin, His6, digoxigenin, FITC, Cy3, Cy5, green fluorescent protein, V5 epitope tags, GST, β-galactosidase, AU1, AU5, and avidin. Half-life extenders include Fc domain and serum albumin.

The term “linked to”, “anchored” or “associated with” is understood in the present disclosure as any interaction between two groups, for example, an interaction between a polypeptide with a GPI group or an interaction between a GPI anchored polypeptide with a membrane. This includes enzymatic interaction, ionic binding, covalent binding, non-covalent binding, hydrogen bonding, London forces, Van der Waals forces, hydrophobic interaction, lipophilic interactions, magnetic interactions, electrostatic interactions, and the like.

The term “loading” or “loading extracellular vesicles” is understood in the present disclosure as an activity or status to result that the vesicles comprise one or more molecules of interest normally not present therein inside, within, and/or on their membrane surface of the vesicles. In some embodiments, the cargo molecules are loaded in the lumen of the extracellular vesicles. In some embodiments, the cargo molecules are loaded onto the outer surface of the extracellular vesicles. In some embodiments, the cargo molecules are loaded within the membrane of the extracellular vesicles.

The term “nucleic acid molecule” refers to a single or double-stranded polymer of deoxyribonucleotide or ribonucleotide bases. It includes chromosomal DNA and self-replicating plasmids, vectors, mRNA, tRNA, siRNA, etc. which may be recombinant and from which exogenous polypeptides may be expressed when the nucleic acid is introduced into a cell.

The term “polypeptide” or “peptide” is understood in the present disclosure as a sequence of amino acids made up of amino acids joined by peptide bonds. The term may be used interchangeably with “protein” in its broadest sense to refer to a molecule of two or more amino acids, amino acid analogs, or peptidomimetics. In some cases, the amino acids are linked by peptide bonds. In some cases, the amino acids are linked by other types of bonds, e.g., ester, ether, etc. As used herein the term “amino acid” refers to either natural and/or unnatural or synthetic amino acids, including glycine and both the D or L optical isomers, and amino acid analogs and peptidomimetics.

In some cases, peptides or polypeptides of the present disclosure contain at least two amino acid residues and are less than about 50 amino acids (for example, 40 amino acids, 30 amino acids, 20 amino acids, or any numbers therein) in length. In some cases, peptides or polypeptides of the present disclosure contain at least 50 amino acids, 100 amino acids, 150 amino acids, or more. In some cases, a peptide or polypeptide is provided with a counterion. In some embodiments, a peptide or polypeptide comprises a N- and/or C-terminal modification such as a blocking modification that reduced degradation or possesses a post-translationally linked GPI group.

The terms “purified”, “isolated”, and “harvested” are used interchangeably and are intended to mean having been removed from its natural environment. The terms purified or isolated does not require absolute purity or isolation; rather, it is intended as a relative term.

The term “vector” is a nucleic acid molecule, preferably self-replicating, which transfers and/or replicates an inserted nucleic acid molecule, such as a transgene or exogenous nucleic acid into and/or between host cells. It includes a plasmid or viral chromosome into whose genome a fragment of recombinant DNA is inserted and used to introduce recombinant DNA, or a transgene, into a polypeptide of the present disclosure.

Unless defined otherwise, all technical and scientific terms used herein have the same meaning as commonly understood by one of the ordinary skill in the art to which this invention belongs. Although any methods and materials similar or equivalent to those described herein can be used in the practice or testing of the formulations or unit doses herein, some methods and materials are now described. Unless mentioned otherwise, the techniques employed or contemplated herein are standard methodologies. The materials, methods and examples are illustrative only and not limiting.

### Overview

Disclosed here are methods of enhancing the production of extracellular vesicles (EVs) that are derived or secreted from genetically engineered mammalian EV producer cells. The EVs of the present disclosure can be incorporated with a sufficient amount of one or more therapeutic agents or any molecules of interest to deliver an effective amount of the therapeutics or any molecules of interest to a target site.

To date, various manufacturing strategies have been contemplated to increase the production of EVs in cells. Some of the strategies include hypoxia induction, tetraspanin protein overexpression, and hypoxia-inducible factor-la overexpression. Genetic modification of genes (e.g., Nad B, SCD4, STEAP3) in some exosome producer cells was previously contemplated for the EV production in cells, although the overall effect of overexpressing these genes were unimpressive. Here, the present disclosure provides highly efficient and superior EV manufacturing methods that enhance the production of EVs in exosome producer cells.

### Methods for Enhancing Extracellular Vesicles (EVs) Production

In one aspect, the disclosure provides a method of enhancing extracellular vesicles (EVs) production that comprises the steps of a) genetically engineering a producer cell to overexpress at least one or more polypeptides and b) harvesting a plurality of EVs from the producer cell.

In some cases, the EVs are ectosomes, exosomes, microvesicles, apoptotic bodies, or any combination thereof. In some cases, the EVs are exosomes.

In some cases, the producer cell is genetically modified to contain the one or more polypeptides. In some cases, the producer cell naturally contains the one or more polypeptides and exosomes derived therefrom also contain the polypeptides. The levels of any desired polypeptides can be modified directly on the exosome (e.g., by contacting the complex with recombinantly produced polypeptides to bring about insertion in or conjugation to the membrane of the complex). Alternatively, or in addition, the levels of any desired polypeptides can be modified directly on the producer cell (e.g., by contacting the complex with recombinantly produced polypeptides to bring about insertion in or conjugation to the membrane of the cell). Alternatively, the producer cell can be modified by transfecting an exogenous nucleic acid into the producer cell to express a desired polypeptide. The polypeptides can already be naturally present on the producer cell, in which case the exogenous construct can lead to overexpression of the polypeptide and increased concentration of the polypeptide in or on the producer cell. Alternatively, a naturally expressed protein can be removed from the producer cell (e.g., by inducing gene silencing in the producer cell). The polypeptides can confer different functionalities to the exosome (e.g., specific targeting capabilities, delivery functions (e.g., fusion molecules), enzymatic functions, increased or decreased half-life in vivo, etc).

In some cases, the polypeptides include, but are not limited to LFA-3, NCAM, PH-20, CD9, CD14, CD16b, CD40, CD46, CD47, CD52, CD55 (DAF), CD58, CD59, CD63, CD81, CD109, GPC1, GPC4, GPC6, CD133, Thy-1, Qa-2, integrins, selectins, lectins, cadherins, carcinoembryonic antigen (CEA), scrapie prion protein, folate-binding protein, or any one of the polypeptides listed in Table A, or any combination thereof.

In some cases, the EVs of the present disclosure are exosomes that comprise one or more polypeptides on its surface selected from LFA-3, NCAM, PH-20, CD9, CD14, CD16b, CD40, CD46, CD47, CD52, CD55 (DAF), CD58, CD59, CD63, CD81, CD109, GPC1, GPC4, GPC6, CD133, Thy-1, Qa-2, carcinoembryonic antigen (CEA), scrapie prion protein, folate-binding protein, or any one of the polypeptide listed in Table A, or any combination thereof. In some cases, the exosome is modified to contain the one or more polypeptides.

In some cases, the producer cell is a mammalian cell. In some cases, the producer cell is selected from the human embryonic kidney 293 cell (HEK293), fibrosarcoma HT-1080 cell, human embryonic retinal PER. C6 cell, kidney/B cell hybrid HKB-11 cell, primary human amniocyte CAP cell, human mesenchymal stem cell (MSC), or hepatoma HuH-7 human cell. In some cases, the producer cell is HEK293-H, HEK293-T, HEK293-EBNA1, or HEK293-F. In some cases, the producer cell is genetically engineered to provide transient overexpression of the polypeptide. In some cases, the producer cell is a genetically engineered stable cell line that constitutively overexpressing the polypeptide.

In some cases, the polypeptide comprises a glycosyl-phosphatidyl-inositol (GPI) group. In some cases, the GPI group is added post-translationally at the C-terminus of the polypeptide. The GIP is a lipid moiety comprising a phosphoethanolamine linker, glycan core, and phospholipid tail. In some cases, the GPI group is covalently attached to a polypeptide as a post-translational modification marker to allow in lipid raft partitioning, signal transduction, cellular communication, or apical membrane targeting. In some cases, the GPI group addition allows the modified polypeptides to anchor in the outer leaflet of a membrane region. In some cases, the GPI group anchored polypeptides are sorted into exosomes. In some cases, the GPI anchored polypeptides are exposed on the surface of exosomes.

In some cases, the GPI anchored polypeptide is any one of the polypeptide listed in Table A. In some embodiments, the GPI anchored polypeptide is any one or more polypeptides selected from Table A.

In some cases, the producer cell is genetically engineered to overexpress any one of the polypeptides listed in Table A or a functional polypeptide fragment thereof in an amount or copy number sufficient to reside in circulation for 1 day, 2 days, 3 days, 4 days, 5 days, 6 days, 7 days, 8 days, 9 days, 10 days, 11 days, 12 days, 13 days, 14 days or longer. In some embodiments, the producer cell is genetically engineered to overexpress the polypeptide of Table A or functional polypeptide fragments thereof in an amount, copy number and/or ratio sufficient to reside in circulation for 15 days, 21 days, 30 days, 45 days, 60 days, 100 days, 120 days, or longer.

In some cases, the GPI anchored polypeptide is selected from the group consisting of CD52, CD55, CD58, CD59, CD109, GPC1, GPC4, and GPC6.

In some cases, the GPI anchored polypeptide is CD55, a 70 kDa membrane protein also known as complement decay-accelerating factor or DAF. CD55 recognizes C4b and C3b fragments of the complement system that are created during C4 (classical complement pathway and lectin pathway) and C3 (alternate complement pathway) activation. CD55 may block the formation of membrane attack complexes or prevent lysis by the complement cascade. In some cases, the producer cell is genetically engineered to overexpress CD55 polypeptide or a functional polypeptide fragment thereof in an amount or copy number sufficient to reside in circulation for 1 day, 2 days, 3 days, 4 days, 5 days, 6 days, 7 days, 8 days, 9 days, 10 days, 11 days, 12 days, 13 days, 14 days or longer. In some embodiments, the producer cell is genetically engineered to overexpress CD55 polypeptide or functional polypeptide fragments thereof in an amount, copy number and/or ratio sufficient to reside in circulation for 15 days, 21 days, 30 days, 45 days, 60 days, 100 days, 120 days, or longer.

In some cases, the GPI anchored polypeptide is CD59, also known as MAC-inhibitory protein (MAC-IP), membrane inhibitor of reactive lysis (MIRL), protectin, or HRF is a protein that attaches to host cells via a glycophosphatidylinositol (GPI) anchor. In some cases, the producer cell is genetically engineered to overexpress CD59 polypeptide or a functional polypeptide fragment thereof in an amount or copy number sufficient to reside in circulation for 1 day, 2 days, 3 days, 4 days, 5 days, 6 days, 7 days, 8 days, 9 days, 10 days, 11 days, 12 days, 13 days, 14 days or longer. In some embodiments, the producer cell is genetically engineered to overexpress CD59 polypeptide or functional polypeptide fragments thereof in an amount, copy number and/or ratio sufficient to reside in circulation for 15 days, 21 days, 30 days, 45 days, 60 days, 100 days, 120 days, or longer.

In some cases, the GPI anchored polypeptide is CD52. CD52 is expressed at high levels on T and B lymphocytes and lower levels on monocytes while being absent on granulocytes and bone marrow precursors. In some cases, the producer cell is genetically engineered to overexpress CD52 polypeptide or a functional polypeptide fragment thereof in an amount or copy number sufficient to reside in circulation for 1 day, 2 days, 3 days, 4 days, 5 days, 6 days, 7 days, 8 days, 9 days, 10 days, 11 days, 12 days, 13 days, 14 days or longer. In some embodiments, the producer cell is genetically engineered to overexpress CD52 polypeptide or functional polypeptide fragments thereof in an amount, copy number and/or ratio sufficient to reside in circulation for 15 days, 21 days, 30 days, 45 days, 60 days, 100 days, 120 days, or longer.

In some cases, the GPI anchored polypeptide is selected from the group consisting of CD52, CD55, CD58, and CD59. In some cases, the polypeptide comprises a glycosyl-phosphatidyl-inositol (GPI) group, an extracellular domain, a transmembrane domain, cytoplasmic domain, or a combination thereof.

In some cases, the producer cell is genetically engineered by transfecting a recombinant vector system to overexpress the polypeptide. The term, “transfection” or “to transfect” as used herein refers to a method of introducing exogenous genetic material into a host cell (e.g., mammalian cell, via lentivirus) wherein the host cell may be transiently transfected or stably transfected. The genetic material may be an expression vector comprising a gene of interest (e.g., a recombinant GPI anchored polypeptide) or a polynucleotide sequence encoding siRNA or shRNA. It also may refer to the introduction of a viral nucleic acid sequence in a way which is for the respective virus the naturally one. The viral nucleic acid sequence needs not to be present as a naked nucleic acid sequence but may be packaged in a viral protein envelope.

Transfection of eukaryotic host cells with a polynucleotide or expression vector, resulting in genetically modified cells or transgenic cells, can be performed by any method known in the art (see e.g., Sambrook J, et al., 1989. Molecular Cloning: A Laboratory Manual. Cold Spring Harbor: Cold Spring Harbor Laboratory Press). Transfection methods include, but are not limited to liposome-mediated transfection, calcium phosphate co-precipitation, electroporation, nucleofection, nucleoporation, microporation, polycation (such as DEAE-dextran)-mediated transfection, protoplast fusion, viral infections and microinjection. The transformation may result in a transient or stable transformation of the host cells. In some cases, the transfection is a stable transfection. In some cases, the transfection is a transient transfection. The transfection method that provides optimal transfection frequency and expression of the heterologous genes in the particular host cell line and type is favored. Suitable methods can be determined by routine procedures. For stable transfectants, the constructs are either integrated into the host cell's genome or an artificial chromosome/mini-chromosome or located episomally so as to be stably maintained within the host cell. Thus, the stably transfected sequences actually remain in the genome of the cell and its daughter cells. Typically, this involves the use of a selectable marker gene and the gene of interest or the polynucleotide sequence encoding the RNA is integrated together with the selectable marker gene. The cells possessing such selectable marker genes are screened and selected for further cultivation (including passaging, growing, culturing, splitting at an optimal cell density). In some cases, the entire expression vector integrates into the cell's genome, in other cases only parts of the expression vector integrate into the cell's genome. Cells “stably expressing” a recombinant polypeptide or an RNA is stably transfected with a gene encoding said recombinant polypeptide or with a polynucleotide sequence encoding said RNA. Thus, the sequences encoding the recombinant polypeptide or RNA remain in the genome of the cell and its daughter cells.

In some cases, the recombinant vector system comprises a nucleic acid sequence encoding a GPI anchored polypeptide. In some cases, the nucleic acid sequence encodes a portion of the GPI anchored protein. In some cases, the nucleic acid sequence encodes an N-terminal domain of the GPI anchored protein. In some cases, the nucleic acid sequence encodes a C-terminal domain of the GPI anchored protein. In some cases, the nucleic acid sequence encodes N- and C-terminal domains of the GPI anchored protein.

In some cases, the nucleic acid sequence encodes the N-terminal domain of any one of the polypeptides listed in Table A. In some cases, the nucleic acid sequence encodes the C-terminal domain of any one of the polypeptides listed in Table A. In some cases, the nucleic acid sequence encodes the N-terminal and C-terminal domain of any one of the polypeptides listed in Table A. In some cases, the nucleic acid sequence encodes the N-terminal domain of CD52. In some cases, the nucleic acid sequence encodes the C-terminal domain of CD52. In some cases, the nucleic acid sequence encodes N- and C-terminal domains of CD52. In some cases, the nucleic acid sequence encodes the N-terminal domain of CD55. In some cases, the nucleic acid sequence encodes the C-terminal domain of CD55. In some cases, the nucleic acid sequence encodes N- and C-terminal domains of CD55. In some cases, the nucleic acid sequence encodes the N-terminal domain of CD58. In some cases, the nucleic acid sequence encodes the C-terminal domain of CD58. In some cases, the nucleic acid sequence encodes N- and C-terminal domains of CD58. In some cases, the nucleic acid sequence encodes the N-terminal domain of CD59. In some cases, the nucleic acid sequence encodes the C-terminal domain of CD59. In some cases, the nucleic acid sequence encodes N- and C-terminal domains of CD59. In some cases, the nucleic acid sequence encodes the N-terminal domain of CD46. In some cases, the nucleic acid sequence encodes the C-terminal domain of CD46. In some cases, the nucleic acid sequence encodes N- and C-terminal domains of CD46.

In some cases, the nucleic acid sequence encodes the amino acid sequence selected from Table 1.

TABLE 1

| Name | Amino acid sequences |
|---|---|
| CD55 | MTVARPSVPAALPLLGELPRLLLLVLLCLPAVWGDCGLPPDVPNAQPALEGRTSFPE |
| | DTVITYKCEESFVKIPGEKDSVICLKGSQWSDIEEFCNRSCEVPTRLNSASLKQPYITQ |
| | NYFPVGTVVEYECRPGYRREPSLSPKLTCLQNLKWSTAVEFCKKKSCPNPGEIRNGQ |
| | IDVPGGILFGATISFSCNTGYKLFGSTSSFCLISGSSVQWSDPLPECREIYCPAPPQIDNG |
| | IIQGERDHYGYRQSVTYACNKGFTMIGEHSIYCTVNNDEGEWSGPPPECRGKSLTSK |
| | VPPTVQKPTTVNVPTTEVSPTSQKTTTKTTTPNAQATRSTPVSRTTKHFHETTPNKGS |
| | GTTSGTTRLLSGHTCFTLTGLLGTLVTMGLLT* |
| CD59 | MGIQGGSVLFGLLLVLAVFCHSGHSLQCYNCPNPTADCKTAVNCSSDFDACLITKAG |
| | LQVYNKCWKFEHCNFNDVTTRLRENELTYYCCKKDLCNFNEQLENGGTSLSEKTVL |
| | LLVTPFLAAAWSLHP* |
| CD46 | MEPPGRRECPEPSWRFPGLLLAAMVLLLYSFSDACEEPPTFEAMELIGKPKPYYEIGE |
| | RVDYKCKKGYFYIPPLATHTICDRNHTWLPVSDDACYRETCPYIRDPLNGQAVPAN |
| | GTYEFGYQMHFICNEGYYLIGEEILYCELKGSVAIWSGKPPICEKVLCTPPPKIKNGK |
| | HTFSEVEVFEYLDAVTYSCDPAPGPDPFSLIGESTIYCGDNSVWSRAAPECKVVKCRF |
| | PVVENGKQISGFGKKFYYKATVMFECDKGFYLDGSDTIVCDSNSTWDPPVPKCLKV |
| | LPPSSTKPPALSHSVSTSSTTKSPASSASGPRPTYKPPVSNYPGYPKPEEGILDSLDVW |
| | VIAVIVIAIVVGVAVICVVPYRYLQRRKKKGTYLTDETHREVKFTSL* |
| CD52 | MKRFLFLLLTISLLVMVQIQTGLSGQNDTSQTSSPSASSNISGGIFLFFVANAIIHLFCFS |
| | * |
| HA-CD55, #4 | MTVARPSVPAALPLLGELPRLLLLVLLCLPAVWGDYPYDVPDYAGSGSGSDCGLPP |
| | DVPNAQPALEGRTSFPEDTVITYKCEESFVKIPGEKDSVICLKGSQWSDIEEFCNRSCE |
| | VPTRLNSASLKQPYITQNYFPVGTVVEYECRPGYRREPSLSPKLTCLQNLKWSTAVE |
| | FCKKKSCPNPGEIRNGQIDVPGGILFGATISFSCNTGYKLFGSTSSFCLISGSSVQWSDP |
| | LPECREIYCPAPPQIDNGIIQGERDHYGYRQSVTYACNKGFTMIGEHSIYCTVNNDEG |
| | EWSGPPPECRGKSLTSKVPPTVQKPTTVNVPTTEVSPTSQKTTTKTTTPNAQATRSTP |
| | VSRTTKHFHETTPNKGSGTTSGTTRLLSGHTCFTLTGLLGTLVTMGLLT* |
| HA-CD55, #3 | MTVARPSVPAALPLLGELPRLLLLVLLCLPAVWGDYPYDVPDYAGSGSGSSCEVPTR |
| | LNSASLKQPYITQNYFPVGTVVEYECRPGYRREPSLSPKLTCLQNLKWSTAVEFCKK |
| | KSCPNPGEIRNGQIDVPGGILFGATISFSCNTGYKLFGSTSSFCLISGSSVQWSDPLPEC |
| | REIYCPAPPQIDNGIIQGERDHYGYRQSVTYACNKGFTMIGEHSIYCTVNNDEGEWS |
| | GPPPECRGKSLTSKVPPTVQKPTTVNVPTTEVSPTSQKTTTKTTTPNAQATRSTPVSR |
| | TTKHFHETTPNKGSGTTSGTTRLLSGHTCFTLTGLLGTLVTMGLLT* |
| HA-CD55, #2 | MTVARPSVPAALPLLGELPRLLLLVLLCLPAVWGDYPYDVPDYAGSGSGSKSCPNP |
| | GEIRNGQIDVPGGILFGATISFSCNTGYKLFGSTSSFCLISGSSVQWSDPLPECREIYCP |
| | APPQIDNGIIQGERDHYGYRQSVTYACNKGFTMIGEHSIYCTVNNDEGEWSGPPPEC |
| | RGKSLTSKVPPTVQKPTTVNVPTTEVSPTSQKTTTKTTTPNAQATRSTPVSRTTKHFH |
| | ETTPNKGSGTTSGTTRLLSGHTCFTLTGLLGTLVTMGLLT* |
| HA-CD55, #1 | MTVARPSVPAALPLLGELPRLLLLVLLCLPAVWGDYPYDVPDYAGSGSGSIYCPAPP |
| | QIDNGIIQGERDHYGYRQSVTYACNKGFTMIGEHSIYCTVNNDEGEWSGPPPECRGK |
| | SLTSKVPPTVQKPTTVNVPTTEVSPTSQKTTTKTTTPNAQATRSTPVSRTTKHFHETT |
| | PNKGSGTTSGTTRLLSGHTCFTLTGLLGTLVTMGLLT* |
| HA-CD55, #0 | MTVARPSVPAALPLLGELPRLLLLVLLCLPAVWGDYPYDVPDYAGSGSGSKSLTSK |
| | VPPTVQKPTTVNVPTTEVSPTSQKTTTKTTTPNAQATRSTPVSRTTKHFHETTPNKGS |
| | GTTSGTTRLLSGHTCFTLTGLLGTLVTMGLLT* |
| mCherry- | MTVARPSVPAALPLLGELPRLLLLVLLCLPAVWGDVSKGEEDNMAIKEFMRFKVH |
| CD55(Short) | MEGSVNGHEFEIEGEGEGRPYEGTQTAKLKVTKGGPLPFAWDILSPQFMYGSKAYV |
| | KHPADIPDYLKLSFPEGFKWERVMNFEDGGVVTVTQDSSLQDGEFIYKVKLRGTNFP |
| | SDGPVMQKKTMGWEASSERMYPEDGALKGEIKQRLKLKDGGHYDAEVKTTYKAK |
| | KPVQLPGAYNVNIKLDITSHNEDYTIVEQYERAEGRHSTGGMDELYKSGLRSRAQAS |
| | KSLTSKVPPTVQKPTTVNVPTTEVSPTSQKTTTKTTTPNAQATRSTPVSRTTKHFHET |
| | TPNKGSGTTSGTTRLLSGHTCFTLTGLLGTLVTMGLLT* |
| mCherry- | MTVARPSVPAALPLLGELPRLLLLVLLCLPAVWGDVSKGEEDNMAIKEFMRFKVH |
| CD55(Long) | MEGSVNGHEFEIEGEGEGRPYEGTQTAKLKVTKGGPLPFAWDILSPQFMYGSKAYV |
| | KHPADIPDYLKLSFPEGFKWERVMNFEDGGVVTVTQDSSLQDGEFIYKVKLRGTNFP |
| | SDGPVMQKKTMGWEASSERMYPEDGALKGEIKQRLKLKDGGHYDAEVKTTYKAK |
| | KPVQLPGAYNVNIKLDITSHNEDYTIVEQYERAEGRHSTGGMDELYKSGLRSRAQAS |
| | CGLPPDVPNAQPALEGRTSFPEDTVITYKCEESFVKIPGEKDSVICLKGSQWSDIEEFC |
| | NRSCEVPTRLNSASLKQPYITQNYFPVGTVVEYECRPGYRREPSLSPKLTCLQNLKW |
| | STAVEFCKKKSCPNPGEIRNGQIDVPGGILFGATISFSCNTGYKLEGSTSSFCLISGSSV |
| | QWSDPLPECREIYCPAPPQIDNGIIQGERDHYGYRQSVTYACNKGFTMIGEHSIYCTV |
| | NNDEGEWSGPPPECRGKSLTSKVPPTVQKPTTVNVPTTEVSPTSQKTTTKTTTPNAQ |
| | ATRSTPVSRTTKHFHETTPNKGSGTTSGTTRLLSGHTCFTLTGLLGTLVTMGLLT* |
| mCherry- | MEPPGRRECPFPSWRFPGLLLAAMVLLLYSFSDACVSKGEEDNMAIIKEFMRFKVH |
| CD46(Short) | MEGSVNGHEFEIEGEGEGRPYEGTQTAKLKVTKGGPLPFAWDILSPQFMYGSKAYV |
| | KHPADIPDYLKLSFPEGFKWERVMNFEDGGVVTVTQDSSLQDGEFIYKVKLRGTNEP |
| | SDGPVMQKKTMGWEASSERMYPEDGALKGEIKQRLKLKDGGHYDAEVKTTYKAK |
| | KPVQLPGAYNVNIKLDITSHNEDYTIVEQYERAEGRHSTGGMDELYKSGLRSRAQAS |
| | LPPSSTKPPALSHSVSTSSTTKSPASSASGPRPTYKPPVSNYPGYPKPEEGILDSLDVW |
| | VIAVIVIAIVVGVAVICVVPYRYLQRRKKKGTYLTDETHREVKFTSL* |
| mCherry- | MEPPGRRECPFPSWRFPGLLLAAMVLLLYSFSDACVSKGEEDNMAIIKEFMRFKVH |
| CD46(Long) | MEGSVNGHEFEIEGEGEGRPYEGTQTAKLKVTKGGPLPFAWDILSPQFMYGSKAYV |
| | KHPADIPDYLKLSFPEGFKWERVMNEEDGGVVTVTQDSSLQDGEFIYKVKLRGTNFP |
| | SDGPVMQKKTMGWEASSERMYPEDGALKGEIKQRLKLKDGGHYDAEVKTTYKAK |
| | KPVQLPGAYNVNIKLDITSHNEDYTIVEQYERAEGRHSTGGMDELYKSGLRSRAQAS |
| | EEPPTFEAMELIGKPKPYYEIGERVDYKCKKGYFYIPPLATHTICDRNHTWLPVSDDA |
| | CYRETCPYIRDPLNGQAVPANGTYEFGYQMHFICNEGYYLIGEEILYCELKGSVAIWS |
| | GKPPICEKVLCTPPPKIKNGKHTFSEVEVFEYLDAVTYSCDPAPGPDPFSLIGESTIYC |
| | GDNSVWSRAAPECKVVKCRFPVVENGKQISGFGKKFYYKATVMFECDKGFYLDGS |
| | DTIVCDSNSTWDPPVPKCLKVLPPSSTKPPALSHSVSTSSTTKSPASSASGPRPTYKPP |
| | VSNYPGYPKPEEGILDSLDVWVIAVIVIAIVVGVAVICVVPYRYLQRRKKKGTYLTD |
| | ETHREVKFTSL* |
| mOrange- | MVSKGEENNMAIIKEFMRFKVRMEGSVNGHEFEIEGEGEGRPYEGFQTAKLKVTKG |
| CD9 | GPLPFAWDILSPQFTYGSKAYVKHPADIPDYFKLSFPEGFKWERVMNFEDGGVVTVT |
| | QDSSLQDGEFIYKVKLRGTNFPSDGPVMQKKTMGWEASSERMYPEDGALKGEIKM |
| | RLKLKDGGHYTSEVKTTYKAKKPVQLPGAYIVGIKLDITSHNEDYTIVEQYERAEGR |
| | HSTGGMDELYKSGLRSRAQASPVKGGTKCIKYLLFGFNFIFWLAGIAVLAIGLWLRF |
| | DSQTKSIFEQETNNNNSSFYTGVYILIGAGALMMLVGFLGCCGAVQESQCMLGLFFG |
| | FLLVIFAIEIAAAIWGYSHKDEVIKEVQEFYKDTYNKLKTKDEPQRETLKATHYALNC |
| | CGLAGGVEQFISDICPKKDVLETFTVKSCPDAIKEVFDNKFHLIGAVGIGIAVVMIFGM |
| | IFSMILCCAIRRNREMV* |
| mOrange- | MVSKGEENNMAIIKEFMRFKVRMEGSVNGHEFEIEGEGEGRPYEGFQTAKLKVTKG |
| CD63 | GPLPFAWDILSPQFTYGSKAYVKHPADIPDYFKLSFPEGFKWERVMNFEDGGVVTVT |
| | QDSSLQDGEFIYKVKLRGTNFPSDGPVMQKKTMGWEASSERMYPEDGALKGEIKM |
| | RLKLKDGGHYTSEVKTTYKAKKPVQLPGAYIVGIKLDITSHNEDYTIVEQYERAEGR |
| | HSTGGMDELYKSGLRSRAQASAVEGGMKCVKFLLYVLLLAFCACAVGLIAVGVGA |
| | QLVLSQTIIQGATPGSLLPVVIIAVGVFLFLVAFVGCCGACKENYCLMITFAIFLSLIML |
| | VEVAAAIAGYVFRDKVMSEFNNNERQQMENYPKNNHTASILDRMQADEKCCGAAN |
| | YTDWEKIPSMSKNRVPDSCCINVTVGCGINFNEKAIHKEGCVEKIGGWLRKNVLVV |
| | AAAALGIAFVEVLGIVFACCLVKSIRSGYEVM* |
| mOrange- | MVSKGEENNMAIIKEFMRFKVRMEGSVNGHEFEIEGEGEGRPYEGFQTAKLKVTKG |
| CD81 | GPLPFAWDILSPQFTYGSKAYVKHPADIPDYFKLSFPEGFKWERVMNFEDGGVVTVT |
| | QDSSLQDGEFIYKVKLRGTNFPSDGPVMQKKTMGWEASSERMYPEDGALKGEIKM |
| | RLKLKDGGHYTSEVKTTYKAKKPVQLPGAYIVGIKLDITSHNEDYTIVEQYERAEGR |
| | HSTGGMDELYKSGLRSRAQASGVEGCTKCIKYLLFVFNFVFWLAGGVILGVALWLR |
| | HDPQTTNLLYLELGDKPAPNTFYVGIYILIAVGAVMMFVGFLGCYGAIQESQCLLGT |
| | FFTCLVILFACEVAAGIWGFVNKDQIAKDVKQFYDQALQQAVVDDDANNAKAVVK |
| | TFHETLDCCGSSTLTALTTSVLKNNLCPSGSNIISNLFKEDCHQKIDDLFSGKLYLIGIA |
| | AIVVAVIMIFEMILSMVLCCGIRNSSVY* |
| Nluc-CD46 | MEPPGRRECPFPSWRFPGLLLAAMVLLLYSESDACVFTLEDFVGDWRQTAGYNLDQ |
| Short | VLEQGGVSSLFQNLGVSVTPIQRIVLSGENGLKIDIHVIIPYEGLSGDQMGQIEKIFKV |
| | VYPVDDHHFKVILHYGTLVIDGVTPNMIDYFGRPYEGIAVFDGKKITVTGTLWNGN |
| | KJIDERLINPDGSLLFRVTINGVTGWRLCERILASGLRSRAQASLPPSSTKPPALSHSVS |
| | TSSTTKSPASSASGPRPTYKPPVSNYPGYPKPEEGILDSLDVWVIAVIVIAIVVGVAVI |
| | CVVPYRYLQRRKKKGTYLTDETHREVKFTSL* |
| CD46Short- | MEPPGRRECPFPSWRFPGLLLAAMVLLLYSFSDACSGLRSRAQASLPPSSTKPPALSH |
| Nluc | SVSTSSTTKSPASSASGPRPTYKPPVSNYPGYPKPEEGILDSLDVWVIAVIVIAIVVGV |
| | AVICVVPYRYLQRRKKKGTYLTDETHREVKFTSLGGGGSVFTLEDFVGDWRQTAGY |
| | NLDQVLEQGGVSSLFQNLGVSVTPIQRIVLSGENGLKIDIHVITPYEGLSGDQMGQIEK |
| | IFKVVYPVDDHHFKVILHYGTLVIDGVTPNMIDYFGRPYEGIAVFDGKKITVTGTLW |
| | NGNKIIDERLINPDGSLLFRVTINGVTGWRLCERILA* |
| HA-CD46Short | MEPPGRRECPFPSWRFPGLLLAAMVLLLYSFSDACYPYDVPDYAGSGSGSLPPSSTK |
| | PPALSHSVSTSSTTKSPASSASGPRPTYKPPVSNYPGYPKPEEGILDSLDVWVIAVIVIA |
| | IVVGVAVICVVPYRYLQRRKKKGTYLTDETHREVKFTSL* |
| CD46Short-HA | MEPPGRRECPFPSWRFPGLLLAAMVLLLYSFSDACLPPSSTKPPALSHSVSTSSTTKSP |
| | ASSASGPRPTYKPPVSNYPGYPKPEEGILDSLDVWVIAVIVIAIVVGVAVICVVPYRYL |
| | QRRKKKGTYLTDETHREVKFTSLGSGSGSYPYDVPDYA* |
| BLAM- | MEPPGRRECPFPSWRFPGLLLAAMVLLLYSESDACHPETLVKVKDAEDQLGARVGY |
| CD46Short | TELDLNSGKILESFRPEERFPMMSTFKVLLCGAVLSRIDAGQEQLGRRIHYSQNDLVE |
| | YSPVTEKHLTDGMTVRELCSAAITMSDNTAANLLLTTIGGPKELTAFLHNMGDHVT |
| | RLDRWEPELNEAIPNDERDITMPVAMATTLRKLLTGELLTLASRQQLIDWMEADKV |
| | AGPLLRSALPAGWFLADKSGAGERGSRGILAALGPDGKPSRIVVIYTTGSQATMDERN |
| | RQIAEIGASLIKHWGSGSGSLPPSSTKPPALSHSVSTSSTTKSPASSASGPRPTYKPPVS |
| | NYPGYPKPEEGILDSLDVWVIAVIVIAIVVGVAVICVVPYRYLQRRKKKGTYLTDET |
| | HREVKFTSL* |
| CD46Short- | MEPPGRRECPFPSWRFPGLLLAAMVLLLYSFSDACLPPSSTKPPALSHSVSTSSTIKSP |
| BLAM | ASSASGPRPTYKPPVSNYPGYPKPEEGILDSLDVWVIAVIVIAIVVGVAVICVVPYRYL |
| | QRRKKKGTYLTDETHREVKFTSLGSGSGSHPETLVKVKDAEDQLGARVGYIELDLN |
| | SGKILESFRPEERFPMMSTFKVLLCGAVLSRIDAGQEQLGRRIHYSQNDLVEYSPVTE |
| | KHLTDGMTVRELCSAAITMSDNTAANLLLTTIGGPKELTAFLHNMGDHVTRLDRWE |
| | PELNEAIPNDERDTTMPVAMATTLRKLLTGELLTLASRQQLIDWMEADKVAGPLLR |
| | SALPAGWFIADKSGAGERGSRGILAALGPDGKPSRIVVIYTTGSQATMDERNRQIAEI |
| | GASLIKHW* |

In some cases, the recombinant vector system comprises an expression control sequence operably linked to the nucleic acid sequence. In some cases, the expression control sequence is a promoter. For example, the nucleic acid sequence corresponding to the polypeptide can be inserted into the recombinant vector containing a promoter sequence compatible with specific RNA polymerases. For example, an exemplary vector may contain T3 and T7 promoter sequence compatible with T3 and T7 RNA polymerase, respectively. Examples for other promoter sequences (exemplified for expression in mammalian cells) are promoters and/or enhancers derived from (CMV) (such as the CMV Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. In some cases, the expression control sequence is a polyadenylation signal, such as BGH polyA, SV40 late or early polyA; alternatively, 3′UTRs of immunoglobulin genes. In some cases, the nucleic acid sequence of the polypeptide can be fused to at least one active domain in the N-terminal and/or C-terminal end, said active domain can be selected from the group consisting of: nuclease (e.g. endonuclease or exonuclease), polymerase, kinase, phosphatase, methylase, demethylase, acetylase, deacetylase, topoisomerase, integrase, transposase, ligase, helicase, recombinase, transcriptional activator (e.g., VP64, VP16), transcriptional inhibitor (e.g., KRAB), DNA end processing enzyme (e.g., Trex2, Tdt), and reporter molecule (e.g. fluorescent proteins, lacZ, luciferase).

In some cases, the recombinant vector system comprises a selection marker. In some cases, the selection marker is Ampicillin, Chloramphenicol, Kanamycin, Tetracycline, Blasticidin S, Neomycin, Hygromycin B, or any combination thereof.

In some cases, the nucleic acid sequence comprises a sequence corresponding to at least one fluorescent marker. In some cases, the fluorescent marker is a green fluorescent protein (e.g., GFP, EGFP, AmCyan, etc.), a red fluorescent protein (e.g., mCherry, DsRed, tdTomato, mStrawberry, etc.), an orange and yellow fluorescent protein (e.g., mOrange, mBanana, ZsYellow, etc.), a Far-red fluorescent protein (e.g., E-Crimson, HcRed, mRasberry, mPlum, etc.), or any combination thereof. In some cases, the fluorescent marker is mCherry.

In some cases, the identity or amount of the producer cells or exosomes can be assessed by in vitro assays. For example, the identity or amount of the producer cells or exosomes is assessed by counting the number of cells or complexes in a population, e.g., by microscopy, by flow cytometry, or by hemacytometry. Alternatively, or in addition, the identity or amount of the producer cells or exosomes is assessed by analysis of protein content of the cell or complex, e.g., by flow cytometry, Western blot, immunoprecipitation, fluorescence spectroscopy, chemiluminescence, mass spectrometry, or absorbance spectroscopy. In some cases, the protein content assayed is a surface protein, e.g., a differentiation marker, a receptor, a co-receptor, a transporter, a glycoprotein. In some embodiments, the identity or amount of the producer cells or exosomes is assessed by analysis of the receiver content of the cell or complex, e.g., by flow cytometry, Western blot, immunoprecipitation, fluorescence spectroscopy, chemiluminescence, mass spectrometry or absorbance spectroscopy. For example, the identity or amount of the producer cells or exosomes can be assessed by the mRNA content of the cells or complexes, e.g., by RT-PCR, flow cytometry or northern blot. The identity or amount of the producer cells can be assessed by nuclear material content, e.g., by flow cytometry, microscopy, or southern blot, using, e.g., a nuclear stain or a nucleic acid probe. Alternatively, or in addition, the identity or amount of the producer cells or exosomes is assessed by lipid content of the cells or complexes, e.g., by flow cytometry, liquid chromatography or by mass spectrometry.

In another aspect, the present disclosure provides a method of making a EV producing stable cell line. In some cases, the method comprises the steps of a) transfecting an EV producer cell with an expression vector, wherein the expression vector comprises a nucleic acid sequence of at least one polypeptide and a selection marker, wherein the at least one polypeptide is linked to a glycosyl-phosphatidyl-ino sitol (GPI) group; b) screening and selecting the transfected cells; and c) cultivating the selected cells.

In some cases, the polypeptide is selected from the group consisting of LFA-3, NCAM, PH-20, CD9, CD14, CD16b, CD40, CD46, CD47, CD52, CD55 (DAF), CD58, CD59, CD63, CD81, CD133, Thy-1, Qa-2, carcinoembryonic antigen (CEA), scrapie prion protein, folate-binding protein, any one of the polypeptides listed in Table A, and any combination thereof. In some cases, the polypeptide is derived from LFA-3, NCAM, PH-20, CD9, CD14, CD16b, CD40, CD46, CD47, CD52, CD55 (DAF), CD58, CD59, CD63, CD81, CD133, Thy-1, Qa-2, carcinoembryonic antigen (CEA), scrapie prion protein, folate-binding protein, any one of the polypeptides listed in Table A, and any combination thereof. In some cases, the polypeptide is derived from CD52, CD55, CD58, or CD59. In some cases, the polypeptide is derived from CD59. In some cases, the polypeptide is derived from CD55. In some cases, the polypeptide is CD59. In some cases, the polypeptide is CD55.

In some cases, the expression vector comprises an expression control sequence operably linked to the nucleic acid sequence. In some cases, the expression control sequence is a promoter. In some cases, the nucleic acid sequence comprises at least one fluorescent marker. In some cases, the selection marker is selected the group consisting of neomycin resistance, puromycin resistance, hygromycin resistance, DHFR resistance, GPT resistance, zeocin resistance, G418 resistance, phleomycin resistance, blasticidin resistance, and histidinol resistance.

In some cases, the EVs are harvested by dialysis or ultra-centrifugation. In some cases, the EVs are harvested by ultra-centrifugation. In some cases, any one of the cells of the present disclosure comprising the EVs may be filtered through a filter with a suitable mesh or pore size (e.g., nylon mesh cell strainers). The filter may have the pore size of 50 nm to 100 mM. In some cases, the filter may have the pore size of 80 nm to 90 mM. In some cases, the filter may have the pore size of 100 nm to 80 mM. In some cases, the filter may have the pore size of about 200 nm to about 70 mM, about 400 nm to about 60 mM, about 600 nm to about 50 mM, about 800 nm to about 40 mM, or about 1 mM to about 20 mM.

In some cases, the concentration of the harvested EVs from the producer cell is 2-fold to 300-fold, 250-fold, 200-fold, 150-fold, or 100-fold higher than those from a control cell (i.e., a wild type cell or a vehicle transfected cell), or any values or ranges therebetween. In some cases, the concentration of the harvested EVs from the producer cell is 2-fold to 90-fold higher than those from a control cell. In some cases, the concentration of the harvested EVs from the producer cell is 2-fold to 80-fold higher than those from a control cell. In some cases, the concentration of the harvested EVs from the producer cell is 2-fold to 70-fold higher than those from a control cell. In some cases, the concentration of the harvested EVs from the producer cell is 2-fold to 60-fold higher than those from a control cell. In some cases, the concentration of the harvested EVs from the producer cell is 2-fold to 50-fold higher than those from a control cell. In some cases, the concentration of the harvested EVs from the producer cell is 2-fold to 40-fold higher than those from a control cell. In some cases, the concentration of the harvested EVs from the producer cell is 2-fold to 30-fold higher than those from a control cell. In some cases, the concentration of the harvested EVs from the producer cell is 2-fold to 20-fold higher than those from a control cell. In some cases, the concentration of the harvested EVs from the producer cell is 2-fold to 10-fold higher than those from a control cell. In some cases, the concentration of the harvested EVs from the producer cell is 2-fold to 8-fold higher than those from a control cell. In some cases, the concentration of the harvested EVs from the producer cell is 2-fold to 4-fold higher than those from a control cell. In some cases, the concentration of the harvested EVs from the producer cell is 2-fold to 2-fold higher than those from a control cell. In some cases, the concentration of the harvested EVs from the producer cell is at least 2-fold higher than those from a control cell. In some cases, the concentration of the harvested EVs from the producer cell is at least 4-fold higher than those from a control cell. In some cases, the concentration of the harvested EVs from the producer cell is at least 6-fold higher than those from a control cell. In some cases, the concentration of the harvested EVs from the producer cell is at least 8-fold higher than those from a control cell. In some cases, the concentration of the harvested EVs from the producer cell is at least 10-fold higher than those from a control cell. In some cases, the concentration of the harvested EVs from the producer cell is at least 15-fold higher than those from a control cell. In some cases, the concentration of the harvested EVs from the producer cell is at least 20-fold higher than those from a control cell. In some cases, the concentration of the harvested EVs from the producer cell is at least 25-fold higher than those from a control cell. In some cases, the concentration of the harvested EVs from the producer cell is at least 30-fold higher than those from a control cell. In some cases, the concentration of the harvested EVs from the producer cell is at least 35-fold higher than those from a control cell. In some cases, the concentration of the harvested EVs from the producer cell is at least 40-fold higher than those from a control cell. In some cases, the concentration of the harvested EVs from the producer cell is at least 45-fold higher than those from a control cell. In some cases, the concentration of the harvested EVs from the producer cell is at least 50-fold higher than those from a control cell.

### Extracellular Vesicles (EVs)

The present disclosure provides a method of enhancing EV production in a mammalian cell.

In some cases, the EVs of the present disclosure are exosomes. In some cases, the exosome comprises a membrane that forms a particle that has a diameter of 30-100 nm, 30-200 nm or 30-500 nm. In some cases, the exosome comprises a membrane that forms a particle that has a diameter of 10-100 nm, 20-100 nm, 30-100 nm, 40-100 nm, 50-100 nm, 60-100 nm, 70-100 nm, 80-100 nm, 90-100 nm, 100-200 nm, 100-150 nm, 150-200 nm, 100-250 nm, 250-500 nm, or 10-1000 nm. In some cases, the EVs have an average diameter length of at least about 180 nm. In some cases, the EVs have an average diameter length of at least about 80 nm to about 180 nm, about 85 nm to about 175 nm, about 90 nm to about 160 nm, about 92 nm to about 150 nm, about 96 nm to about 140 nm, about 98 nm to about 130 nm, about 100 nm to about 120 nm, about 102 nm to about 112 nm, or about 105 nm to about 110 nm. The size of the EVs may change following loading of the cargo molecules. In other cases, the size of the EVs may remain the same after loading. In some cases, the exosomes have an average diameter length of at least about 80 nm. In some cases, the membrane comprises lipids and fatty acids. In some cases, the membrane comprises one or more of phospholipids, glycolipids, fatty acids, sphingolipids, phosphoglycerides, sterols, cholesterols, and phosphatidylserine. In some cases, the membrane can comprise one or more polypeptides and one or more polysaccharides, such as glycans. In some cases, the produced exosomes can have a specific density between 0.5-2.0, 0.6-1.0, 0.7-1.0, 0.8-1.0, 0.9-1.0, 1.0-1.1, 1.1-1.2, 1.2-1.3, 1.4-1.5, 1.0-1.5, 1.5-2.0, and 1.0-2.0 kg/m3.

In some cases, the plurality of EVs may be manufactured, purified or isolated from cells, cell culture medium, or tissues as described in Example 1. In some cases, the plurality of EVs may be purified or isolated prior to contacting a detergent or cargo molecule. In some cases, the plurality of EVs may be purified or isolated after contacting a detergent-removal agent. In some cases, the EVs may be purified or isolated as the plurality of cargo-loaded EVs.

In some cases, the EVs are loaded with cargo molecules. In some cases, the cargo molecules comprise an active pharmaceutical ingredient (API). In some cases, the API comprises small molecule therapeutics. In some cases, the cargo molecule comprises a polypeptide, protein, lipid, nucleic acid, carbohydrate, lipid, metabolite, or any combinations thereof. In some cases, the nucleic acid comprises DNA. In some cases, the nucleic acid comprises peptide nucleic acids (PNAs). In some cases, the nucleic acid comprises RNA. In some cases, wherein the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (lncRNA). In some cases, the protein comprises an antibody or enzyme. In some cases, the cargo molecule comprises antisense oligonucleotide. In some cases, the cargo molecule comprises morpholino oligomer. In some cases, the cargo molecule comprises one or more components of a gene editing system. In some cases, the gene editing system is selected from the group consisting of CRISPR/Cas, zinc finger nuclease, transcription, and activator-like effector nuclease (TALEN).

In yet another aspect, the present disclosure provides a cell line manufactured according to any one of the methods described herein. The present disclosure also provides a kit for enhancing EV production that comprises any one of the producer cells or the cell lines described herein. The present disclosure also provides a composition that comprises a plurality of EVs according to any one of the EVs described herein. In some cases, the composition further comprises a pharmaceutically acceptable excipient.

Disclosed herein are EVs that comprise at least one cargo molecule. Also disclosed herein is a method of manufacturing the EVs for loading a sufficient amount of one or more cargo molecules so that an appropriate amount of the one or more cargo molecules is delivered to or within a target cell or tissue of interest.

In some cases, the one or more cargo molecules have a final concentration of about 0.1 μM to about 300 μM. In some cases, the one or more cargo molecules have a final concentration of at least about 0.1 μM to about 1 μM, about 0.1 μM to about 10 μM, about 0.1 μM to about 20 μM, about 0.1 μM to about 50 μM, about 0.1 μM to about 100 μM, about 0.1 μM to about 150 μM, about 0.1 μM to about 200 μM, about 0.1 μM to about 300 μM, about 1 μM to about 10 μM, about 1 μM to about 20 μM, about 1 μM to about 50 μM, about 1 μM to about 100 μM, about 1 μM to about 150 μM, about 1 μM to about 200 μM, about 1 μM to about 300 μM, about 10 μM to about 20 μM, about 10 μM to about 50 μM, about 10 μM to about 100 μM, about 10 μM to about 150 μM, about 10 μM to about 200 μM, about 10 μM to about 300 μM, about 20 μM to about 50 μM, about 20 μM to about 100 μM, about 20 μM to about 150 μM, about 20 μM to about 200 μM, about 20 μM to about 300 μM, about 50 μM to about 100 μM, about 50 μM to about 150 μM, about 50 μM to about 200 μM, about 50 μM to about 300 μM, about 100 μM to about 150 μM, about 100 μM to about 200 μM, about 100 μM to about 300 μM, about 150 μM to about 200 μM, about 150 μM to about 300 M, or about 200 μM to about 300 μM. In some cases, the one or more cargo molecules have a total concentration of at least about 0.1 μM, about 1 μM, about 10 μM, about 20 μM, about 50 μM, about 100 μM, about 150 μM, about 200 μM, or about 300 μM.

In some cases, the transfection or overexpression of the polypeptide produces low or no toxicity in any one of the cells or kits of the present disclosure. In some cases, the cells, EVs, exosomes, or kits of the present disclosure may comprise a pharmaceutically acceptable detergent. In these cases, the detergent is a nonionic detergent. In such cases, the detergent is Polyethylene glycol p-(1, 1, 3, 3-tetramethylbutyl)-phenyl ether (Triton X-100R). In some cases, the detergent is octaethylene glycol monododecyl ether (OEG). In some cases, the EVs comprise polyethylene glycol p-(1, 1, 3, 3-tetramethylbutyl)-phenyl ether at a final concentration of about 0.03 mM to about 4 mM, about 0.04 mM to about 3 mM, about 0.05 mM to about 2.5 mM, about 0.06 mM to about 2.2 mM, about 0.08 mM to about 2 mM, about 0.1 mM to about 1.8 mM, about 0.2 mM to about 1.5 mM or any concentration in between thereof.

In some cases, the EVs of the present disclosure may form a detergent-mixture solution having a final detergent concentration of about 0.005% v/v to about 10% v/v, about 0.01% v/v to about 9.8% v/v, about 0.02% v/v to about 9.6% v/v, about 0.04% v/v to about 9.4% v/v, about 0.06% v/v to about 9.2% v/v, about 0.08% v/v to about 9.0%, about 0.1% v/v to about 8.0% v/v, about 0.1% v/v to about 7.0% v/v, about 0.1% v/v to about 6.0% v/v, about 0.1% v/v to about 5.0% v/v, about 0.2% v/v to about 4.0% v/v, about 0.4% v/v, about 0.5% v/v, about 0.6% v/v, about 0.8% v/v, about 1.0% or any concentrations in between.

In some cases, the EVs may contact the cargo molecule and the detergent by adding the cargo molecule and the detergent simultaneously. In some cases, the biological sample comprising extracellular vesicles may contact the cargo molecule and the detergent by adding the cargo molecule and the detergent sequentially. In some cases, the method comprises a step of contacting the biological sample comprising a plurality of EVs with the cargo molecule and the detergent by adding the cargo molecule prior to adding the detergent.

In some embodiments, the present disclosure provides a composition comprising any one of the extracellular vesicles (EVs) producer cells of the present disclosure, wherein the EV producer cell is genetically engineered to overexpress at least one or more polypeptides, wherein the polypeptide is linked to a glycosyl-phosphatidyl-inositol (GPI) group. In some cases, the polypeptide is derived from CD52, CD55, CD58, CD59, CD109, GPC1, GPC4, GPC6, or any one of the polypeptide listed in Table A. In some cases, the EVs producer cells comprise a recombinant vector system that comprises a nucleic acid sequence encoding the coding sequence of the polypeptide. In some cases, the recombinant vector system comprises a coding sequence of the polypeptide selected from any one of sequences in Table 1. In some cases, the recombinant vector system comprises a coding sequence of the polypeptide of mCherry-CD46 (Short), HA-CD46Short, or CD46Short-HA of Table 1.

In some cases, the producer cell further comprises a release helper selected from the group consisting of protein G (G) of vesicular stomatatis virus (VSV), glycoprotein B (gB) of Herpes Simplex virus 1 (HSV-1), baculovirus fusion protein gp64, and gB from EpsteinBarr virus (EBV). In some cases, the release helper is Vesicular Stomatitis Virus Glycoprotein (VSVG).

### Method of Using EVs

The present disclosure also provides methods, kits and reagents for using the EVs for treating a disease or disorders in a subject in need thereof. For example, a method of using EVs for treating a patient suffering from chronic and recurrent diseases by administering an effective amount of the EVs to the patient is provided herein. In some cases, the chronic and recurrent diseases may be diabetes, infection, protein deficiencies, or immunological disorders.

In some embodiments, the present disclosure provides a method of delivering a cargo molecule into a target cell, wherein the cargo molecule is delivered by extracellular vesicles, wherein the extracellular vesicles comprising the cargo molecule are produced by any one of the genetically engineered producer cells of the present disclosure. In some embodiments, the producer cell overexpresses at least one or more polypeptides. In some embodiments, the one or more polypeptides is linked to a glycosyl-phosphatidyl-inositol (GPI) group. In some embodiments, the one or more polypeptides is linked to the cargo molecules. In some embodiments, the one or more polypeptides is linked to a glycosyl-phosphatidyl-inositol (GPI) group and a cargo molecule. In some embodiments, the polypeptide is derived from any one of the polypeptide listed in Table A.

In some embodiments, the producer cell is genetically engineered by transfecting a recombinant vector system. In some embodiments, the recombinant vector system comprises a nucleic acid sequence encoding a glycosyl-phosphatidyl-inositol (GPI) group. In some embodiments, the recombinant vector system comprises a nucleic acid sequence encoding a cargo molecule. In some embodiments, the recombinant vector system comprises a nucleic acid sequence encoding a glycosyl-phosphati dyl-inositol (GPI) group and a cargo molecule. In some embodiments, the cargo molecule is a polypeptide, protein, lipid, nucleic acid, carbohydrate, metabolite, or any combinations thereof. In some embodiments, the nucleic acid comprises RNA. In some embodiments, the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (lncRNA). In some embodiments, the protein is an antibody or enzyme.

In some embodiments, the producer cell produces extracellular vesicles that carry the cargo molecule on the outer surface of the extracellular vesicles. In some embodiments, the producer cell produces extracellular vesicles that carry the cargo molecule on the inner surface of the extracellular vesicles. In some embodiments, the producer cell produces extracellular vesicles that carry the cargo molecule in the lumen of the extracellular vesicles.

In the therapeutic method of the present disclosure, the EVs may be administered to the patient via intravenous, intra-arterial, intranasal, or topical administration route. The effective dosage could be evaluated by the attending physician on an empirical basis or set by in vivo or in vitro evaluation for each pathology.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1. Enhanced Extracellular Vesicle (EV) Production in Mammalian Cells

### Cell Culture

Human embryonic kidney 293 cells (HEK 293T) were cultured in DMEM cell medium supplemented with 10% of fetal bovine serum in a humid incubator with 5% CO₂ at 37° C. Prior to suspension adaptation, the cells were seeded at 5×10⁵ viable cells/mL in serum-free medium containing 5% serum (v/v) in a low-binding 6-well plate placed on a shaker at 120 rpm. The cells were then incubated in the 37° C. incubator to achieve maximum cell density of about >85% cell viability, which was considered to be an indication of adequate cell adaptation under the experimental conditions. The adapted cells were passaged via sequential splitting in cell culture medium with decreasing concentrations of serum at each split, from 5% serum to 2%, to 1%, to 0.5%, to 0.1%, and to 0% of serum in medium.

For suspension of 293F cells, the cells were cultured in serum-free medium placed in a 37° C. humid incubator with 5% CO₂, with a constant shaking at 120 rpm.

### Plasmid Construction and Overexpression of Polypeptide of Interest

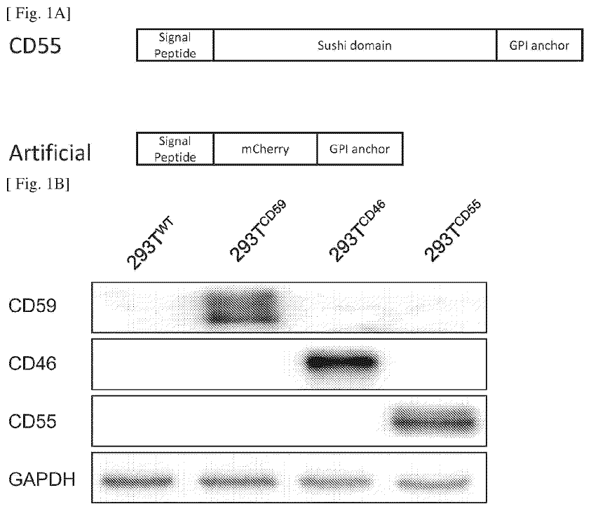

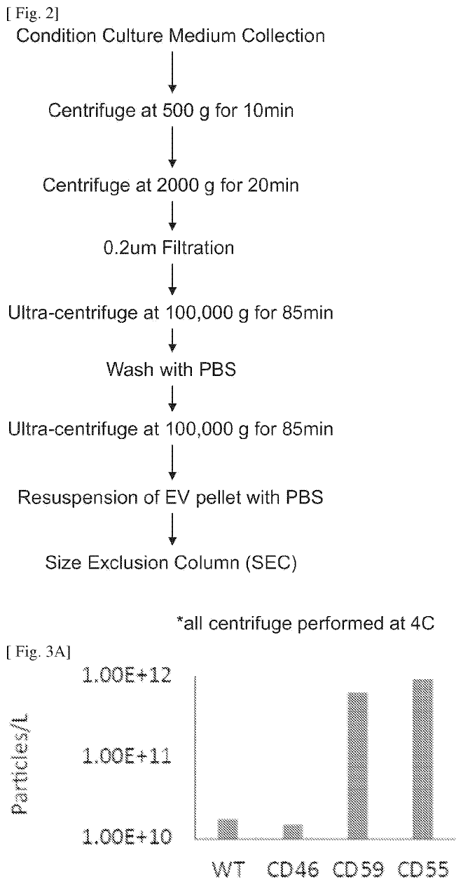

To generate a stable cell line overexpressing the polypeptide of interest, PippyBac (PB) transposon system or Lentivirus system were used. Briefly, a nucleic acid fragment encoding the coding sequence (CDS) of the polypeptide of interest was synthesized and cloned into a PB transposon vector at the BglIl and Xhol restriction enzyme digestion sites or into a pLenti vector at the Xbal and Sall restriction enzyme digestion sites. In this study, the CDS of CD46, CD55, and CD59 were synthesized and cloned into each vector for overexpression. As shown in FIG. 1A, an artificial GPI-anchored protein sequence was also synthesized and cloned into a vector. The artificial sequence of this example comprises mCherry sequence fused with the N- and C-terminal domains of CD55.

Approximately 5×10⁵ of 293T cells were transfected with 2 μg of PB Transposon vector carrying the nucleic acid fragments of the polypeptide in combination with 1 μg of PB Transposase vector by using Lipofectamine 3000, according to the manufacturer's instructions. Or 2×10⁵ of 293T cells were infected with lentivirus containing target genes. The transfected or infected cells were then incubated and selected under 200 μg/mL of hygromycin B for 7-8 days to obtain a stable cell line overexpressing the gene of interest.

### Western Blot

The transfected cells were lysed in cell lysis buffer and the protein concentration of the lysate was measured by the BCA protein assay to ensure equal loading. The samples were resolved by SDS-PAGE, followed by transferring onto a PVDF membrane. The membrane was immunoblotted with anti-CD46 (sc-166159, Santa Cruz Biotechnology), anti-CD55 (sc-51733, Santa Cruz Biotechnology), anti-CD59 (sc-133170, Santa Cruz Biotechnology), and anti-GAPDH (2118S, CST). An updated HRP-linked secondary antibody was used for ECL-based Western blot detection. As shown in FIG. 1B, the overexpressed CD59, CD46, and CD55 protein levels were observed.

### Example 2. Isolation and Quantification of Extracellular Vesicles (EVs)

Small Extracellular Vesicle (sEV) Isolation

Isolation and purification processes of the extracellular exosomes (EVs) followed by quantification are summarized in FIG. 2. Briefly, cells were cultured in Condition cell culture medium. Condition culture medium was harvested into a 50 mL centrifuge tube and immediately subjected to the centrifugation at 500×g for 10 min at 4° C. to remove the liable cells. The supernatant was transferred to a new 50 mL centrifuge tube and was subjected to centrifugation at 2000×g for 20 min at 4° C. to remove dead cells. The supernatant was then filtered through a 0.2-μm membrane to remove cell debris as well as other big extracellular vesicles. The filtered sEVs were pelleted by ultracentrifugation at 100,000×g for 85 min at 4° C. The collected sEVs pellets were washed with PBS and further purified by a second ultracentrifugation at 100,000×g for 85 min at 4° C. The purified pellet of sEVs was resuspended in appropriate volume of PBS then purified with size exclusion column (SEC). The protein concentration of sEVs was quantified by BCA Protein Quantification Assay. The particle concentration of sEVs were measured with Apogee Micro-GxP Flow Cytometer, the productivity (particles/L) was calculated below:

$$Productivity ( \frac{particles}{L} ) = \frac{\begin{matrix}sample concentration ( \frac{particles}{uL} ) \times \\ dilution factor \times sample volume ( uL )\end{matrix}}{condition culture medium harvested ( L )}$$

Nanoparticle tracking analysis (NTA) and Nano flow cytometer (nFCM) were used to measure the size and concentration of the harvested EVs. For NTA measurements, NanoSight NS300 from Malvern Panalytical was used. For nFCM measurements, Micro-GxP flow cytometer from Apogee Flow System was used. In the present disclosure, unless otherwise specified, Micro-G×P system was used to measure the EV particle concentrations in the harvested culture medium.

### Increased EV Production in CD55 and CD59 Overexpressed 293T Cells

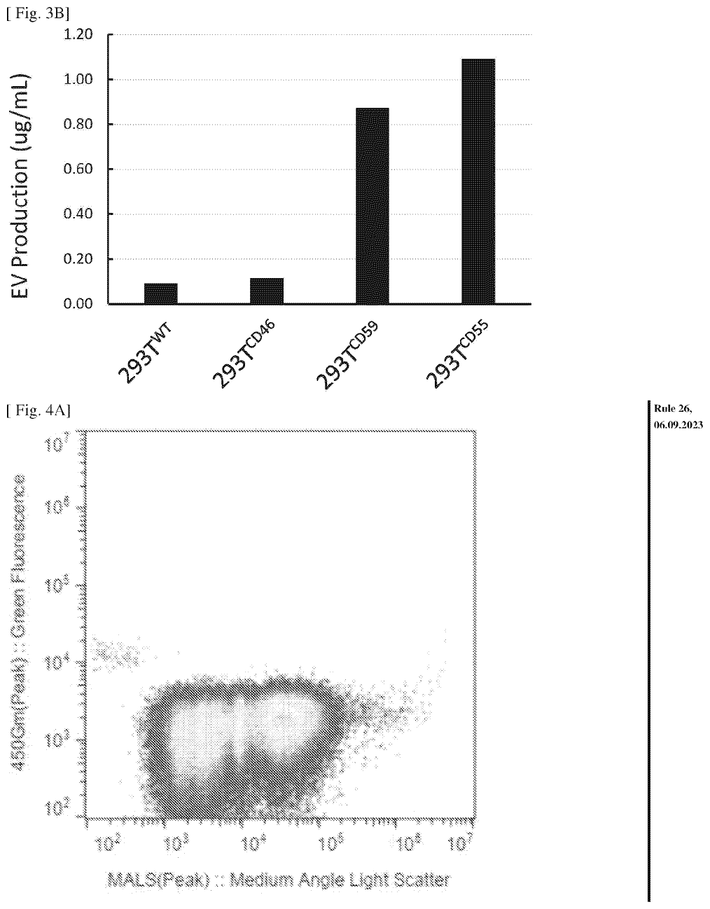

Referring to FIGS. 3A-D, approximately 8- to 40-fold increase in EV production (particles/L or μg/mL) was observed in the CD59 and CD55 overexpressed in 293T cells as compared to vehicle control (293T^{WT}) and/or CD46 overexpressed 293T cells.

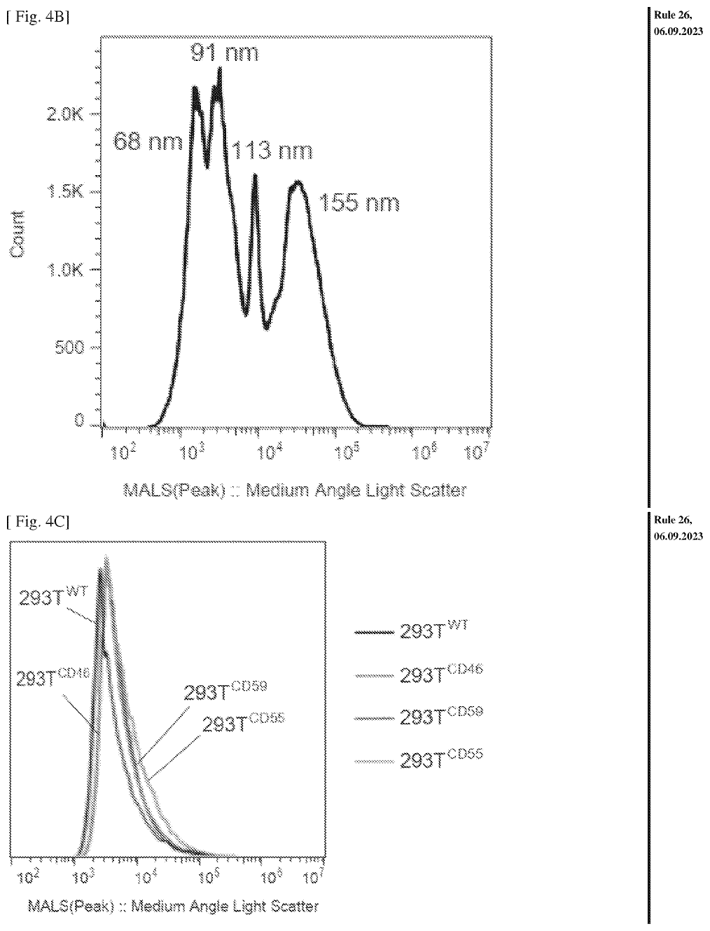

The harvested particles were further characterized to measure the particle size distribution (e.g., <200 nm to be identified as small EV) and study the morphology of the harvested EVs. As shown in FIGS. 4A-C, the particle size was measured by using nano flow cytometer with commercially available silicon beads standard (nFCM silica nanospheres cocktail 1).

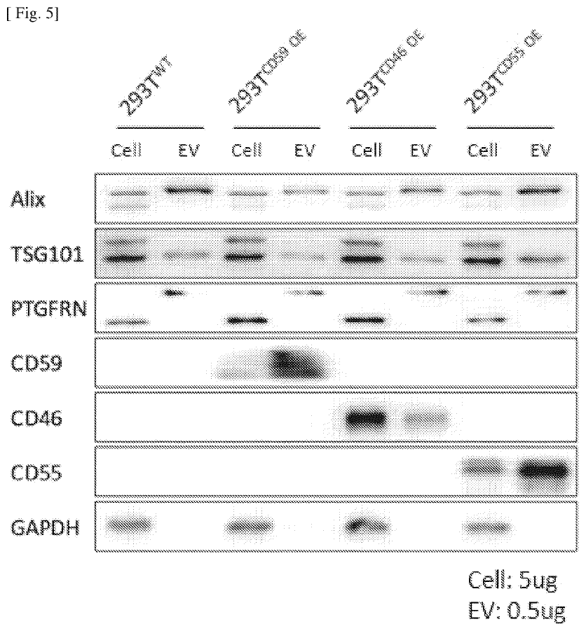

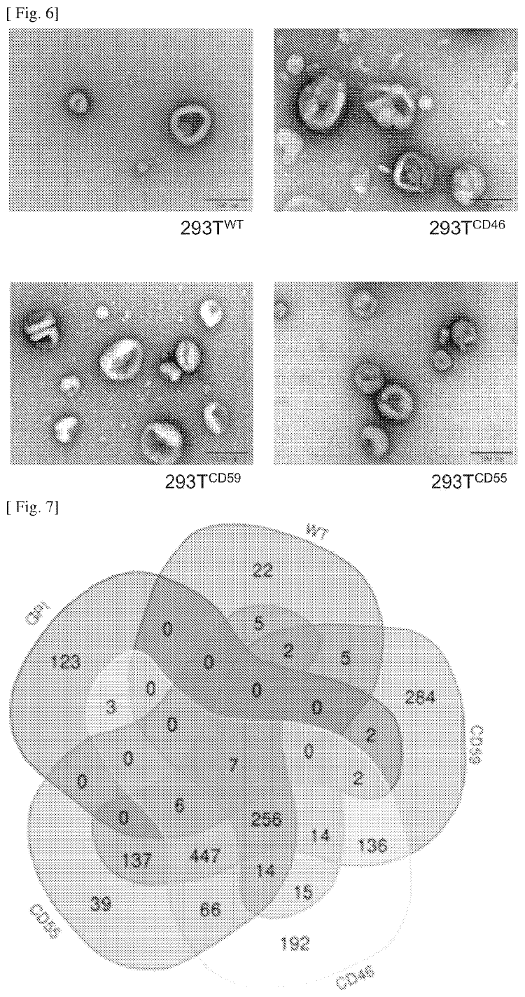

The protein expression levels of CD59, CD46, and CD55 in cells and in EVs following overexpression were measured by Western blotting. As shown in FIG. 5, higher concentrations of CD55 and CD59 were observed in EVs than in total cell lysate. In contrast, overexpressing CD46 resulted in a relatively lower level of CD46 expression in EVs than in cells.

### Transmission Electron Microscopy (TEM)

The morphology and viability of the harvested EVs were observed under TEM. The resuspended purified exosome sample was further diluted to 0.1 μg/μL. An equal volume of 4% paraformaldehyde to the exosome sample was added and incubated for 2 hours. 3 uL of the mixture was dropped onto the TEM grid and fixed with 2% paraformaldehyde for 20 min. The grid was washed with 3×PBS and fixed with 1% glutaraldehyde for 5 min. After 8 times of PBS wash (2 min each) followed by ddH₂O wash (9 times), the gird was stained for 5 min in uranyl oxalate and in 1% methyl cellulose: 4% uranyl acetate (9:1) for 10 min on ice. Excess liquid was removed with a filter paper and the grid was air-dried for 5 to 10 min. Exosomes were examined in a JEOL 1100 transmission electron microscope at 60 kV, and images were obtained with ATM digital camera, as shown in FIG. 6.

### LC-MS/MS

Protein is extracted from cells and EVs samples and quantified with BCA method. Appropriate amount of protein is subjected to enzymatic digestion, and then to liquid chromatography-mass spectrometry (LC-MS/MS) analysis. The raw data was analyzed by MaxQuant to obtain the quantitative results of LFQ and iBAQ.

LFQ is a method for relative protein quantification when comparing multiple samples, the expression of the same protein can be compared. iBAQ is a method of approximate absolute quantification, which can be used to compare the abundance of different proteins in the same sample.

### Example 3. GPI-Anchor Protein Enhancing Extracellular Vesicles (EVs) Production

Table A lists small polypeptides and proteins that are known to possess a glycosyl-phosphatidyl-inositol (GPI) group. Among these, CD55, CD59, GPC1, GPC3, GPC4, GPC6, and SMPDL3B were identified as highly enriched and concentrated GPI-proteins in the EVs harvested from the 293T^{WT}, 293T^{CD46}, 293T^{CD59}, and 293T^{CD55} cells of Example 1 (FIG. 7). All of the identified GPI-anchor proteins (i.e., CD55, CD59, GPC1, GPC3, GPC4, GPC6, and SMPDL3B) were enriched at a higher concentration in the EV than in the total cell fraction, as represented by EV/cell>1 relative protein enrichment quantification in Table 2.

TABLE A
GPI-anchor polypeptides

| | | | | |
|---|---|---|---|---|
| ACHE | CNTN3 | GPC4 | MELTF | RTN4R |
| ALPG | CNTN4 | GPC5 | MMP17 | RTN4RL1 |
| ALPI | CNTN5 | GPC6 | MMP25 | RTN4RL2 |
| ALPL | CNTN6 | GPIHBP1 | MSLN | SEMA7A |
| ALPP | CPM | HFE2 | NCAM1 | SMPDL3B |
| ART1 | CPO | HJV | NEGR1 | SPACA4 |
| ART3 | DPEP1 | HYAL2 | NRN1 | SPAM1 |
| ART4 | DPEP2 | IGSF21 | NRN1L | SPRN |
| BCAN | DPEP3 | ITLN1 | NT5E | TDGF1 |
| BST1 | EFNA1 | IZUMO1R | NTM | TECTA |
| BST2 | EFNA2 | LSAMP | NTNG1 | TECTB |
| CA4 | EFNA3 | LY6D | NTNG2 | TEX101 |
| CD109 | EFNA4 | LY6E | OMG | TFPI |
| CD14 | EENA5 | LY6G6C | OPCML | THY1 |
| CD160 | ENPP6 | LY6G6D | OTOA | TNFRSF10C |
| CD177 | FCGR3B | LY6H | PLAUR | TREH |
| CD24 | FOLR1 | LY6K | PLET1 | ULBP1 |
| CD48 | FOLR2 | LY6L | PRND | ULBP2 |
| CD52 | GAS1 | LY6S | PRNP | ULBP3 |
| CD55 | GFRA1 | LYNX1 | PRSS21 | UMOD |
| CD59 | GFRA2 | LYPD1 | PRSS41 | VNN1 |
| CDH13 | GFRA3 | LYPD2 | PRSS42P | VNN2 |
| CEACAM5 | GFRA4 | LYPD3 | PRSS55 | XPNPEP2 |
| CEACAM6 | GFRalpha-1 | LYPD4 | PSCA | |
| CEACAM7 | GLIPRIL1 | LYPD5 | RAET-1E | |
| CEACAM8 | GML | LYPD6 | RAET1G | |
| CFC1 | GP2 | LYPD6B | RAET1L | |
| CNTFR | GPC1 | LYPD8 | RECK | |
| CNTN1 | GPC2 | MDGA1 | RGMA | |
| CNTN2 | GPC3 | MDGA2 | RGMB | |

TABLE 2
Protein enrichment in EV

| *LFQ: EV/Cell | WT | CD46 | CD55 | CD59 |
|---|---|---|---|---|
| CD55 | 194180001 | 530230001 | 18.3650627 | 172840001 |
| CD59 | 89.0901689 | 470260001 | 196300001 | 8.28679451 |
| GPC1 | 491270001 | 387390001 | 202620001 | 484780001 |
| GPC3 | 91153001 | 565660001 | 196960001 | 1466200001 |
| GPC4 | 838300001 | 2491800001 | 978250001 | 1799800001 |
| GPC6 | 150560001 | 330800001 | 343950001 | 129630001 |
| SMPDL3B | 162910001 | 144220001 | 146630001 | 86740001 |

*LFQ: relative protein quantification. EV enrichment represented as protein detected in EV/Cell >1 while input equal protein amount.

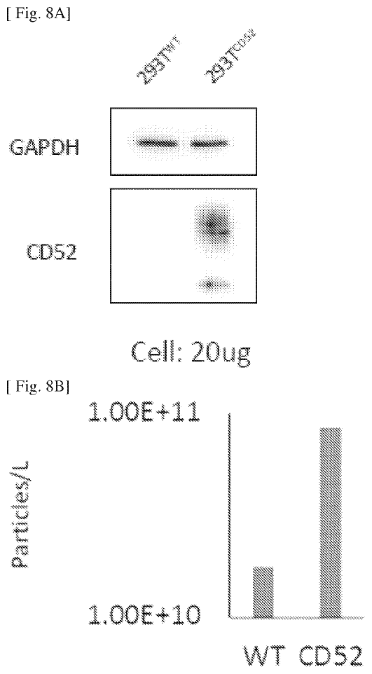

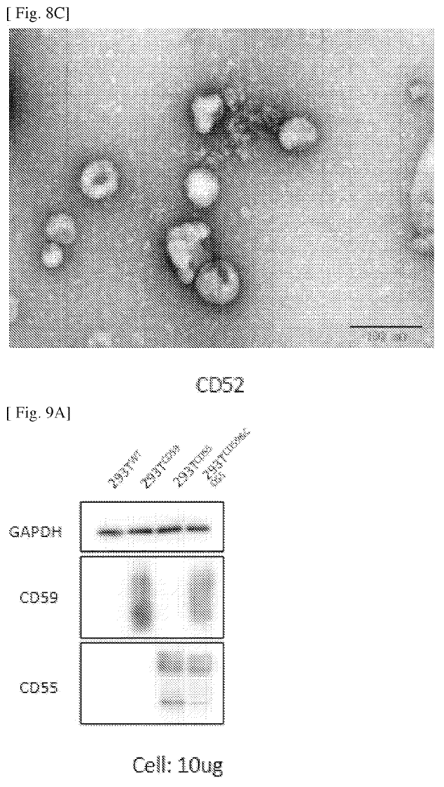

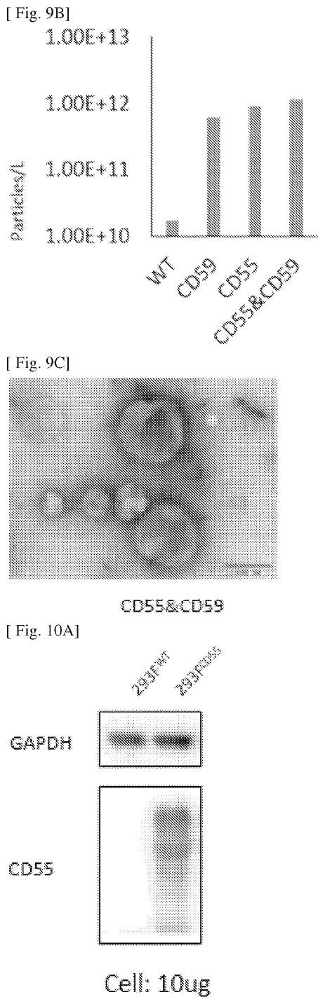

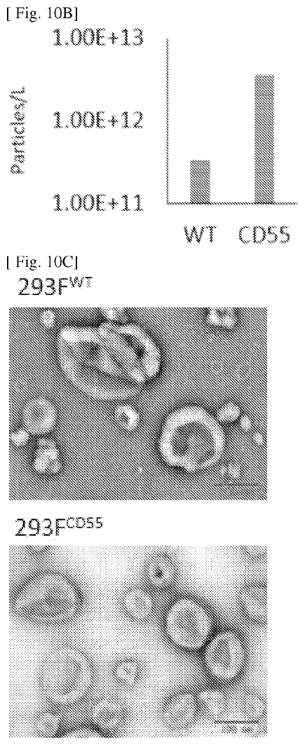

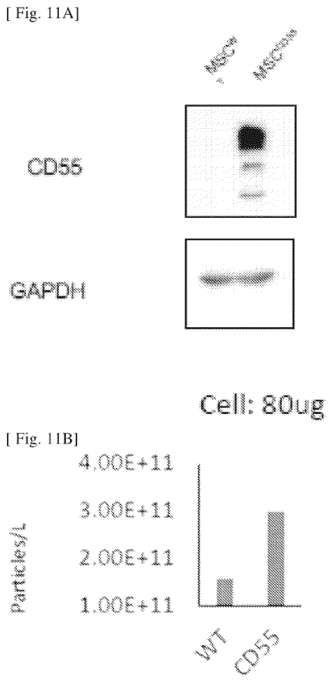

CD52 overexpression also enhanced the EV production in cells (FIGS. 8A-B). The morphology of the harvested EVs from the 293T^{CD52} is shown in FIG. 8C. In addition, a significant increase in the EV production was observed following co-overexpression of the GPI-anchor proteins (i.e., CD55 and CD59) as compared to WT control. However, only a slight increase in EV production was observed compared to when the CD55 and CD59 were separately overexpressed (FIGS. 9A-C).

Similar effects of improvement in EV production of the GPI-anchor protein were observed in other cell lines, such as 293F cells (FIGS. 10A-C) and mesenchymal stem cells (MSC) (FIGS. 11A-C). In the MSC cell-based experiment, human umbilical cord mesenchymal stem cells (hUC-MSCs) were cultured in mesenchymal stem cell basal medium (MSCBM, Dakewe 6114011) supplied with 5% EliteGro-Advance (EPA-050, EliteCell). The CD55 Lentivirus generated with 3-plasmids system was used to infect the MSCs, and MSC^{CD55} cells were selected with 50 ug/mL Hygromycin for stable expression. The selected cells were then cultured in condition medium for 2˜3 days. The culture medium was harvested for EVs isolation.

Mesenchymal stem cells (MSCs) have captured great attention in regenerative medicine for over a few decades by virtue of their differentiation capacity, potent immunomodulatory properties, and their ability to be favorably cultured and manipulated. Recent investigations implied that the pleiotropic effects of MSCs is not associated to their ability of differentiation, but rather is mediated by the secretion of soluble paracrine factors. Extracellular vesicles are one of these paracrine mediators. EVs transfer functional cargos like miRNA and mRNA molecules, peptides, proteins, cytokines and lipids from MSCs to the recipient cells. EVs participate in intercellular communication events and contribute to the healing of injured or diseased tissues and organs. Previous studies reported that EVs alone are responsible for the therapeutic effects of MSCs in numerous experimental models. Based on this, the MSC-derived EVs are to be studied as a novel cell-free therapeutic for treating heart diseases, kidney diseases, liver diseases, immune disorders, and neurological diseases, or promoting cutaneous wound healing.

### Example 4. Overexpression of Glycosyl-Phosphatidyl-Inositol (GPI) Region Improves Extracellular Vesicles (EVs) Production

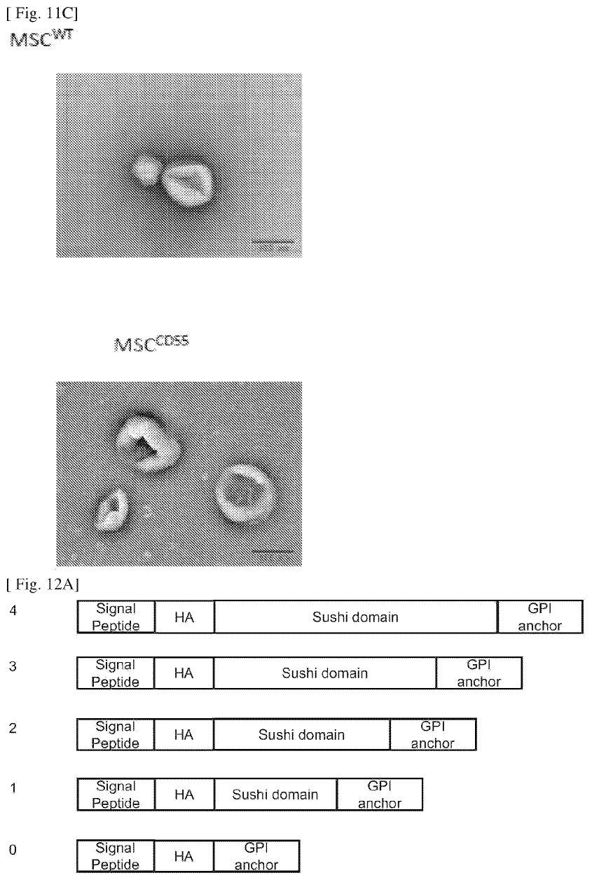

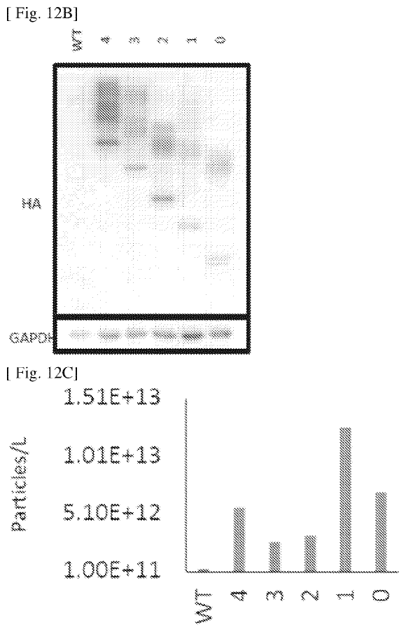

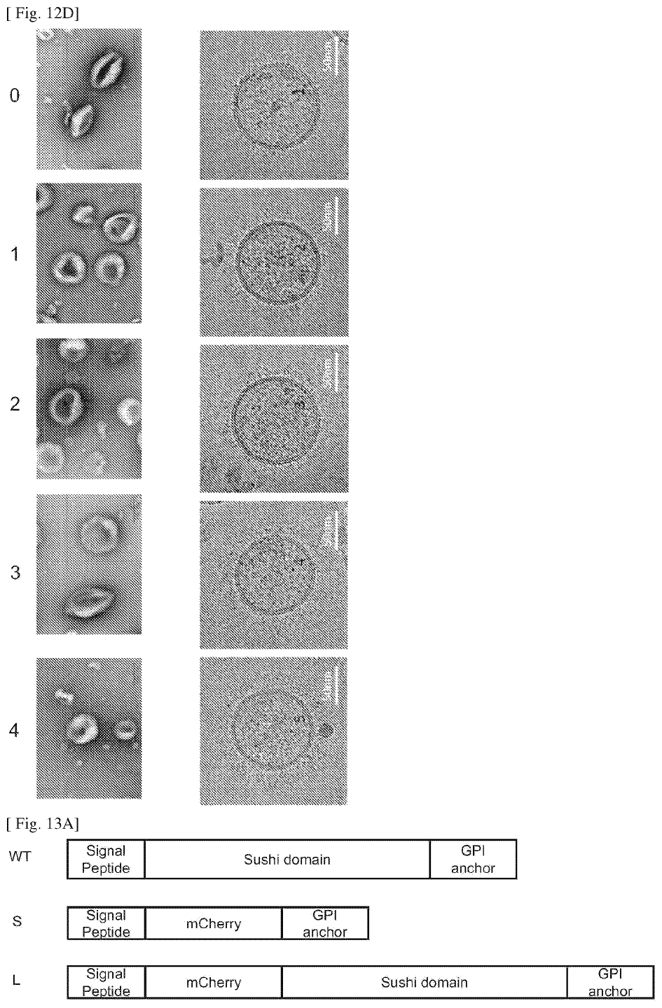

To identify the GPI-anchor protein regions responsible for providing the enhanced EV production, full-length and truncated CD55 constructs (as shown in FIG. 12A) were generated and transfected into the 293F cells. As shown in FIGS. 12B-C, both truncated CD55 (i.e., constructs Nos. 0-3) and full-length CD55 (i.e., construct No. 4) overexpression increased EV production. The morphology of the tested EVs was observed by using Cryo EM (FIG. 12D). Briefly, to prepare samples for cryo-EM, lacey carbon EM grids were glow-discharged. An aliquot (2 μL) of the aqueous solution of the EV sample was applied on to the carbon side of EM grid, which was then blotted and plunge-frozen into the precooled liquid ethanc. The samples were studied in a cryo-electron microscope Talos Arctica.

### Example 5. Effects of GPI-Anchor Proteins on Extracellular Vesicles (EVs) Production

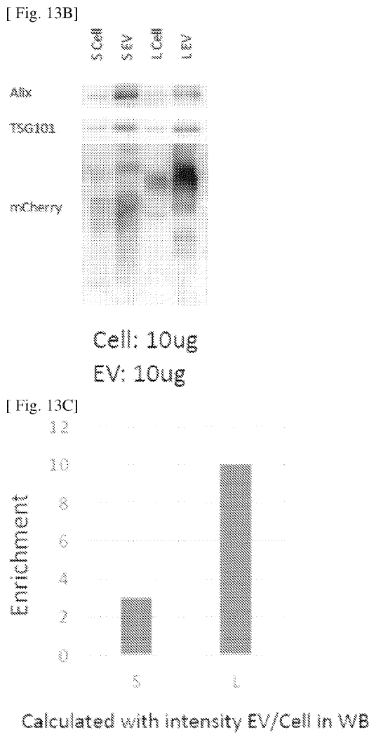

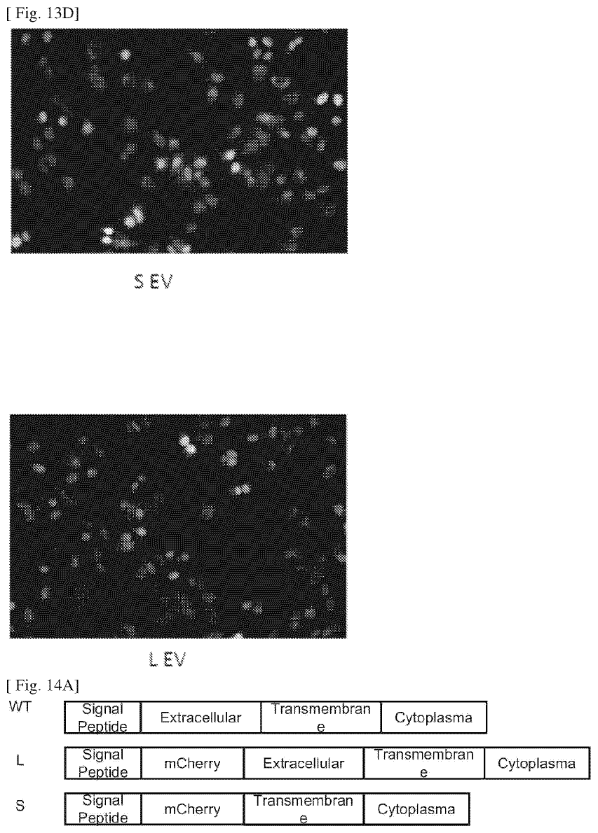

As shown in FIG. 13A, two CD55-based constructs encoded with mCherry (Short: mCherry-CD55-Short and Long: mCherry-CD55-Long) were transfected into 293T cells as described in Example 1. Western blotting was carried out to determine the mCherry (ab213511, Abcam) protein expression levels in the total cell lysate and purified EVs (FIG. 13B). As shown in FIGS. 13B-C, CD55-based GPI-anchor constructs served as scaffolds to load and carry the mCherry sequence onto the EVs. A significantly higher level of mCherry was detected in the EVs derived from the 293T cells transfected with L construct as compared to 293T cells transfected with S construct. The uptake of the mCherry loaded EVs by H2B-GFP expressing target cells were observed with fluorescent microscope (FIG. 13D).

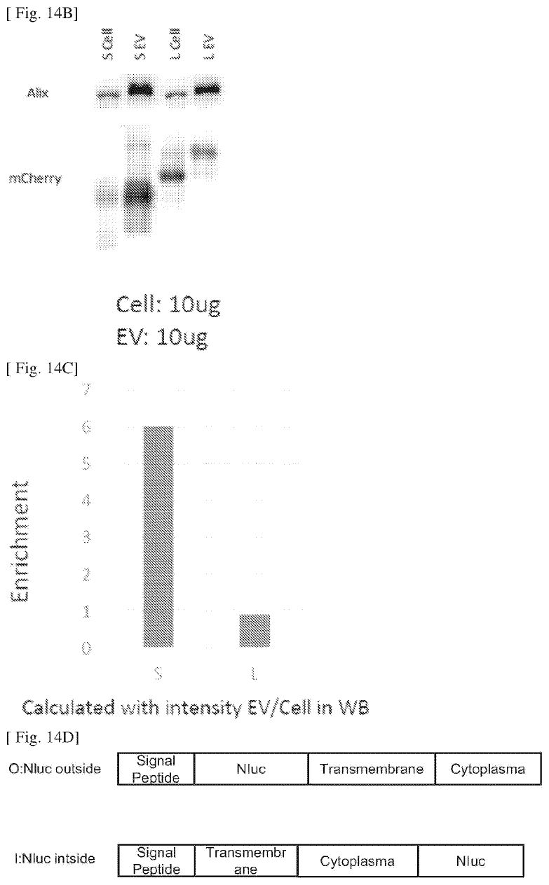

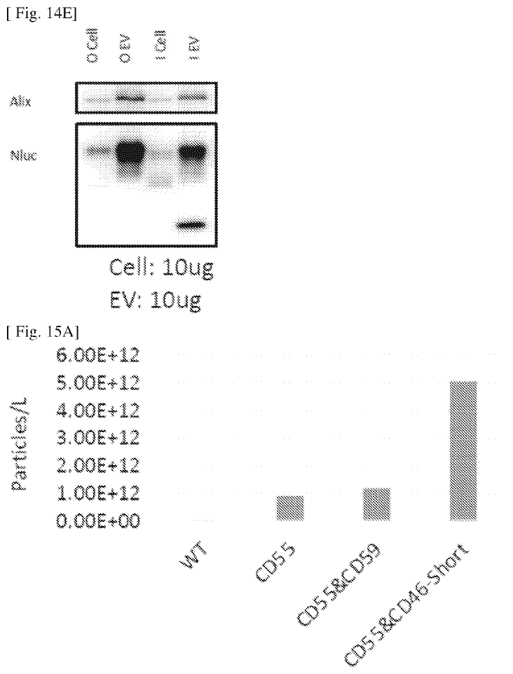

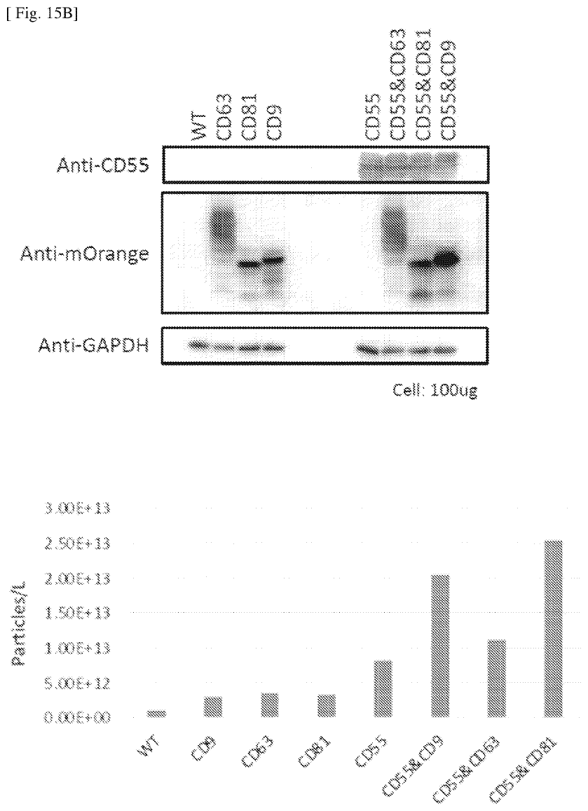

As another example, two CD46-based constructs were fused with mCherry sequence and transfected into 293T cells for overexpression (FIGS. 14A-B). Interestingly, enrichment of the mCherry protein was much higher in the EVs purified from the S construct transfected cells than in the EVs from the L construct transfected cells (FIG. 14C). Considering that the CD46 is a transmembrane protein, two forms of CD46-short construct were generated, each encoding nanoluciferase (Nluc) at a specific position (FIG. 14D, O: Nluc outside and I: Nluc inside). As shown in FIG. 14E, loading of Nluc inside or outside of the EVs was controlled by fusing the Nluc sequence at near N-terminal (outside) or C-terminal (inside, lumen of the EVs).

Interestingly, unlike the CD55 and CD59 co-overexpression, overexpressing CD55 with CD46-short construct resulted even greater increase in the EV production in 293T cells (FIG. 15A). To determine whether other transmembrane proteins would provide the same effect, mOrange tagged transmembrane proteins, i.e., CD9, CD63, and CD81, were overexpressed in the presence of CD55. The data in FIG. 15B show that overexpressing CD9 or CD81 with CD55 resulted synergistic effects in increasing EV production in 293F cells (up to 3-fold increase). The co-expression of CD55 and CD9 or CD81 provided additive effect (2-fold increase).

### Example 6. Preparation of ExoBoost Cells and Extracellular Vesicles (EVs)

For industrial manufacture of ExoBoost cells (i.e., the cells transfected with GPI-anchor proteins), a single colony (colony No. 28) from 293T^{CD55} cells were obtained and grown as 28F cells. The main findings related to upstream and downstream manufacturing processes are summarized below.

### Upstream: Cell Culture

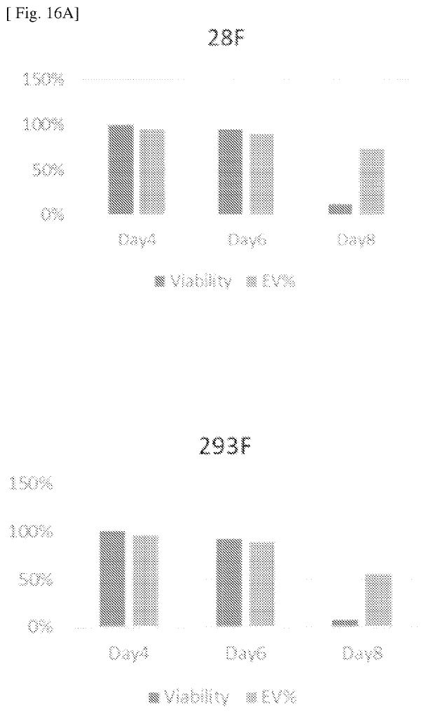

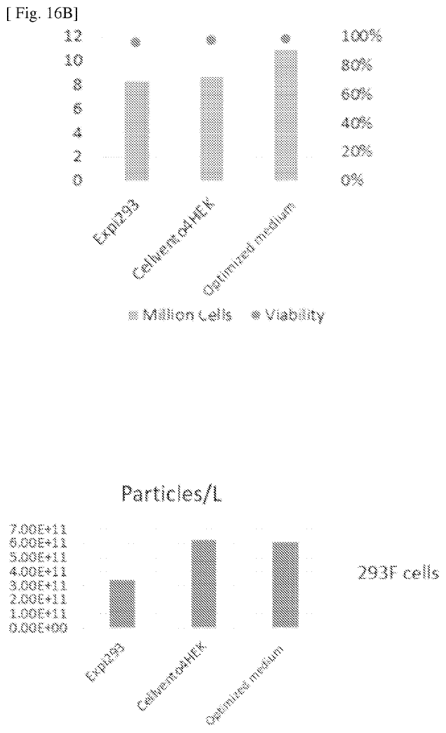

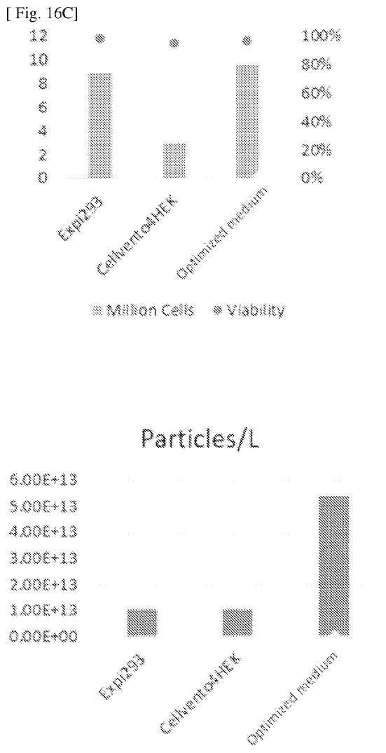

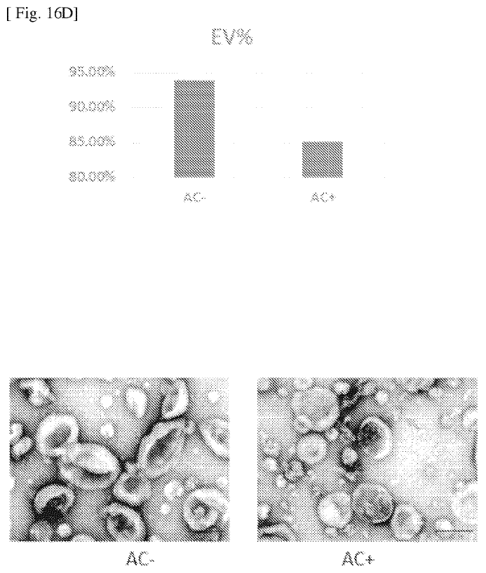

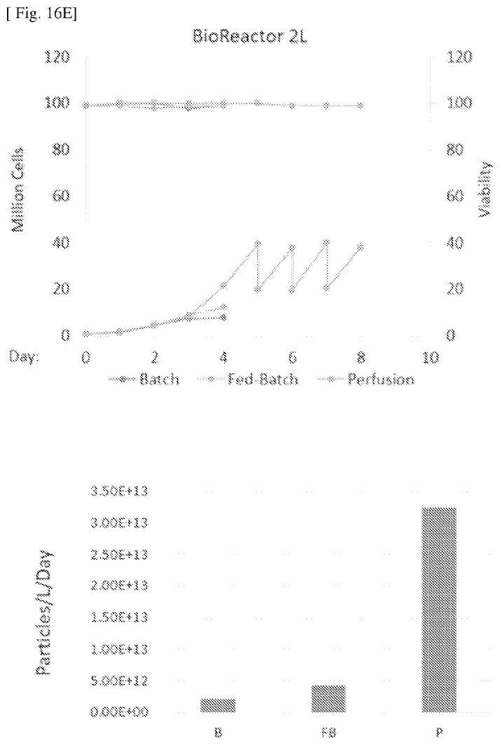

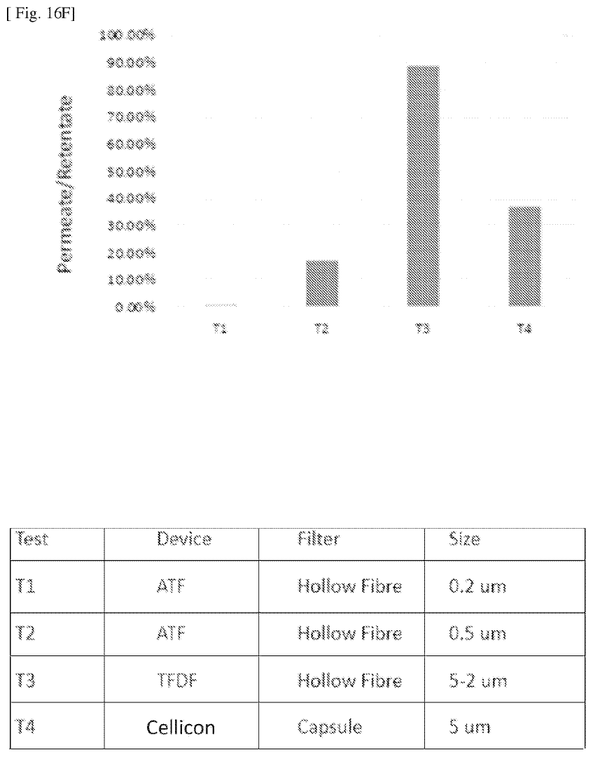

1. As shown in FIG. 16A, the reduction in cell viability decreased the percentage of EV production in 28F and 293F cells.
2. The use of Applicant's optimized culture medium significantly increased the concentration of EVs in both 293F and 28F cells (FIGS. 16B-C).
3. The addition of an anti-clump agent reduced the EV % (FIG. 16D).
4. Compared to batch or fed-batch processes (e.g., one-pot reaction), the perfusion method significantly increased cell viability and EVs production (particles/L/day) (FIG. 16E).
5. In the perfusion phase, a hollow fibre filter with a pore size of 2-5 μm provided a higher permeate/retentate ratio than the hollow filter with smaller pore size or a capsule filter with a pore size of 5 μm (FIG. 16F).

### Downstream: Purification

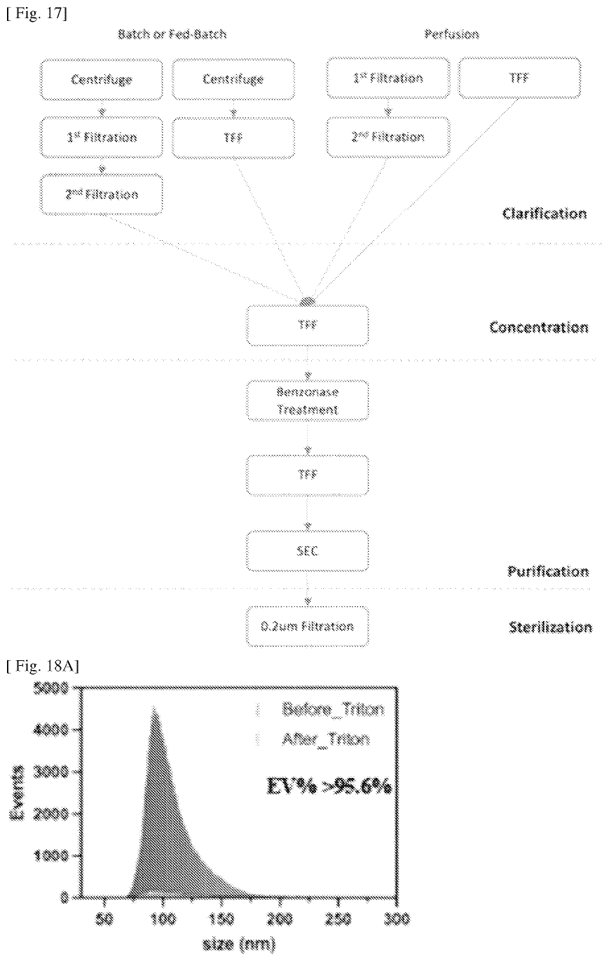

The downstream purification process is summarized in FIG. 17. The 28F cells were cultured with perfusion method. The permeate was clarified with 1st and 2nd filtration then concentrated with TFF. The concentrated sample was treated with benzonase then purified with TFF and SEC. Samples were sterilized by 0.2 um filtration and stored at −80C after dilution to 2 ug/uL and aliquots. All materials used were sterilized in advanced or designed as single-use. Details of quality control method is summarized in Table 3.

TABLE 3
Quality Control

| Attribute | Analysis factor | Methods |
|---|---|---|
| General | Appearance | General observation |
| Properties | Visible particles | General observation |
| | Osmolality | Osmometers |
| | pH | pH meter |
| Safety | Sterility | Direct inoculation |
| | Mycoplasma | qPCR |
| | Endotoxin | Gel Clot |
| | Adventitious Virus | cytopathic effects |
| | | (CPE) |
| | Replication Competent Virus | qPCR |
| Product | Protein Concentration | BCA |
| Specific | Particle Concentration | nFCM |
| | Purity | Calculate Particles/ug |
| | EV % | 0.1% Triton treatment |
| | Size and Distribution | nFCM |
| | Morphology | TEM or cryo-EM |
| | EV Markers | Immunoblotting |
| | Positive markers | |
| | membrane (CD9, CD63, CD81, | |
| | CD82) | |
| | lumen (Alix, TSG101, SDC8P) | |
| | Negative markers | |
| | Golgi (GM130) | |
| | Endoplasmic reticulum (Calnexin) | |
| | Mitochondria (CVC1) | |
| | Content | Omics |
| | Proteomics | LC-MS/MS |
| | Lipidomics | LC-MS/MS |
| | Transcriptomics | NextGen Sequencing |
| Potency | Mechanism of action (MoA) | Based MoA assays |
| Identity | Active pharmaceutical ingredient | Based API assays |
| | (API) | |
| Process | Residual DNA | qPCR |
| Residuals | Benzonase | ELISA |

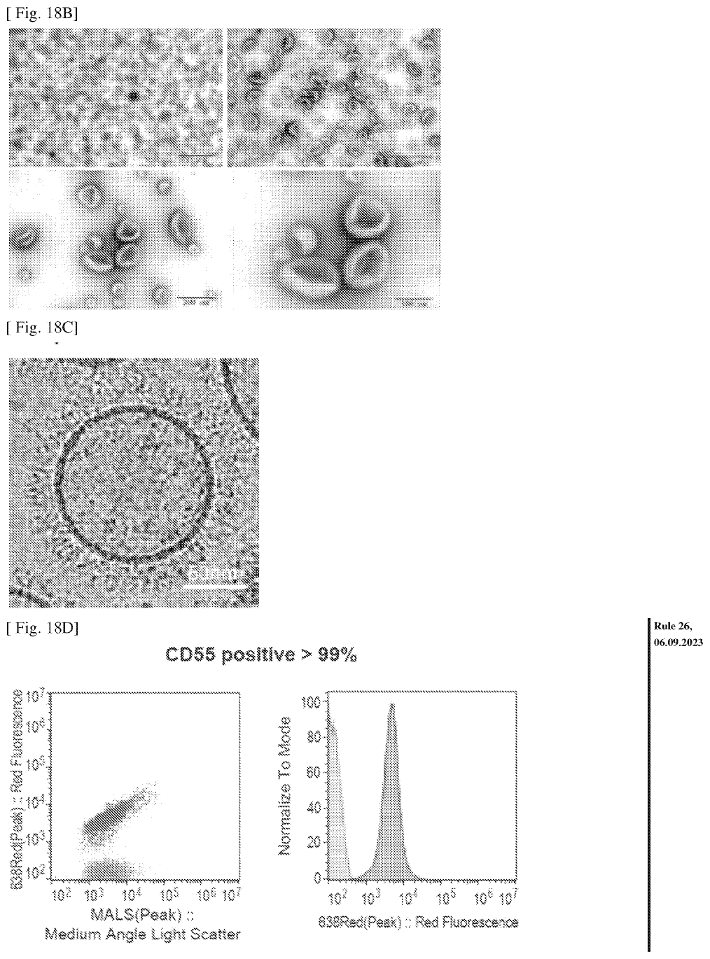

The EVs harvested from 28F cells are labeled as TREXO, and the product quality was confirmed by measuring size distribution and EV % and obtaining TEM, Cryo-EM, nFCM, and immunoblot images (FIGS. 18A-E). For the nFCM results, 5 uL EV sample (2E8 particles/uL) was mixed with 5 uL anti-CD55-APC (10101-R028-A, Sinobiology) or PBS, then incubate at room temperature for 1 hour. The sample was then diluted 500× for nFCM detection.

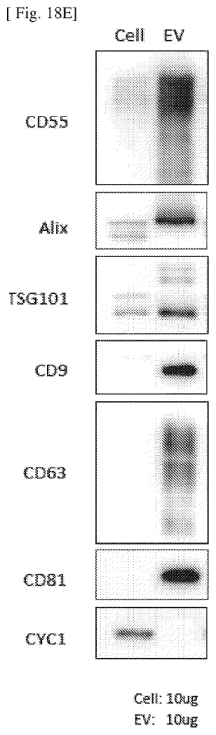

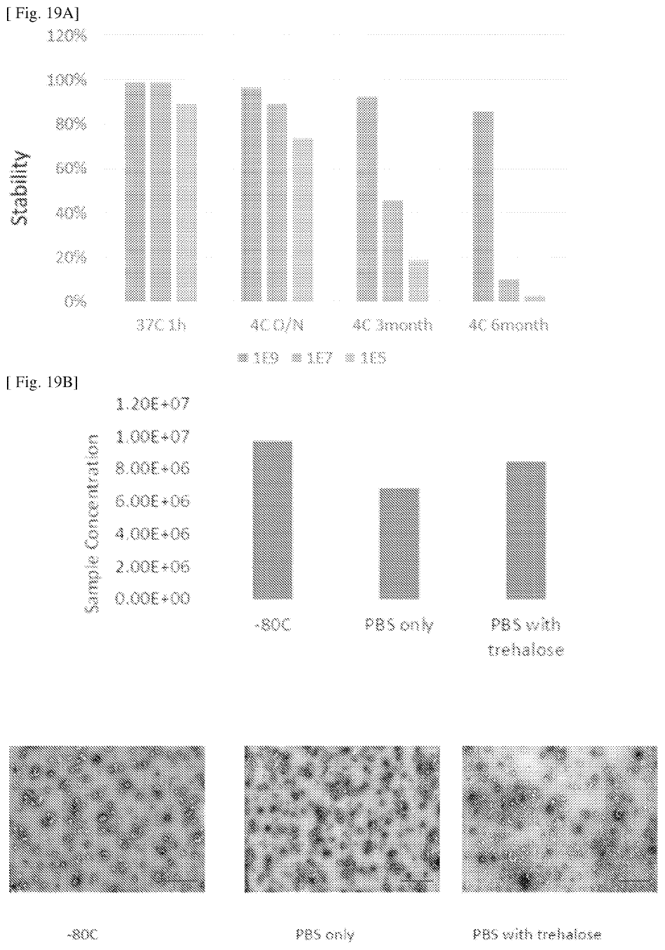

The harvested EVs were stable at 4° C. for 6 months at a concentration higher than 10e9 particles/μL (FIG. 19A) and even longer when stored at −80° C. The stability of the EV samples were also tested under freeze-dry condition. Briefly, the frozen EVs were thawed from −80° C., the particle concentration of thawed sample was measured by nFCM. Then EV samples were diluted to a final concentration of 1e7 particles/uL with either PBS only or 10% (w/w) Trehalose (T0167, Sigma) in PBS. The EVs samples were lyophilized in a freeze dryer (FD-1A-50 LGJ-10, Beijing Song Yuan). The freeze-dried samples were redissolved in ultrapure water with the initial volume prior to lyophilization. EV concentration was tested with nFCM after redissolved, fresh thawed from −80° C. sample as the control. As shown in FIG. 19B, aggregation and degradation of the freeze-dried EVs were observed.

### Example 7. Loading the EVs with Cargo Molecules

Extracellular exosome loading of siRNA as a cargo molecule was studied.

### Loading the Cargo Molecules Outer Portion of the EVs

Cholesterol modified siRNA (GenePharm Co., Ltd) was dissolved in DEPC water without further purification. The purified EV was dispersed in PBS and concentration of the EV particles was determined by nFCM. The EVs and siRNA were mixed by adding the siRNA solutions to the EV suspension with repeated pipetting, then keep the incubation at room temperature for 30 minutes before the downstream purification. A pre-packed chromatography column was used to purify unloaded free siRNA from the final sample. A Cy5-dye modified cholesterol siRNA (GenePharm Co., Ltd) was used for the quantification of loading efficiency and loading capacity. The loading efficiency was measured by nFCM in EV loading %.

Loading the Cargo Molecules into the Lumen of the EVs

Electroporation of EVs were performed by a Gene Pulser Xcell Electroporation System (BioRad) with 0.1-cm cuvettes (#1652089, BioRad). Exponential mode was selected with varied voltage and a fixed capacitance of 125 uF and co resistance. The electroporation buffer was prepared and used fresh at each electroporation experiment. The particle concentration of EVs were adjusted to a final concentration in the range of 1E7 particles/uL to 1E9 particles/uL. After electroporation, the sample was allowed to rest on ice for at least half an hour before purification with a pre-packed chromatography column to remove free RNAs. The loading efficiency and loading capacity were measured by nFCM. To determine whether the RNAs were packed within the lipid membrane of EVs (thus being protected) or on the external surface of EVs, samples after chromatography purification were treated with RNase (RNase A/T1 mixture, Thermo Fischer) or equal volume of PBS (as without RNase control) at 37° C. for 10 minutes. Then samples were measured by nFCM to determine the change of RNA loading efficiency with/without RNase treatment.

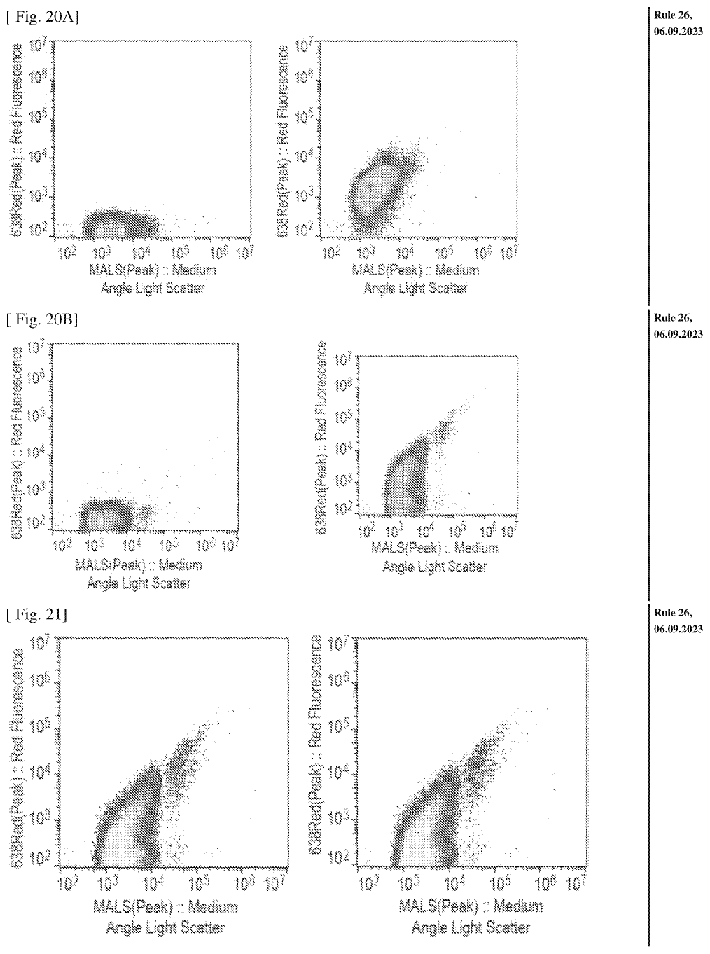

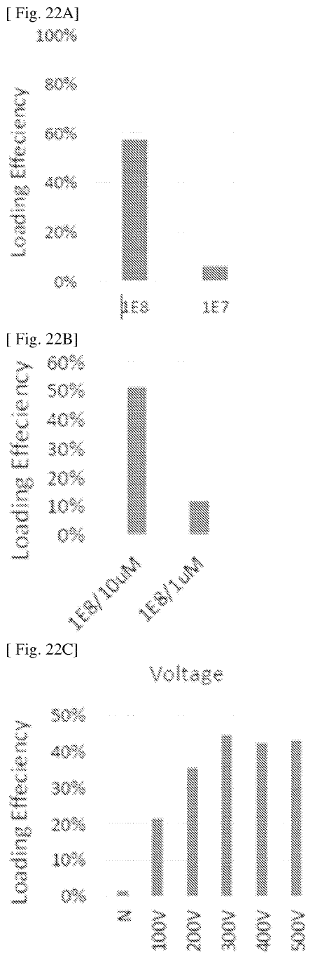

As shown in FIGS. 20A-B, the siRNA loaded outside by hydrophobic modification provided >99% efficiency. About 40-60% efficiency was observed when siRNA was loaded inside of the EVs by electroporation. The loading of the siRNA was further validated by RNAse treatment as shown in FIG. 21. The loading efficiency was affected by multiple factors such as the concentration of the EVs (FIG. 22A), ratio between the concentration of EVs and RNA (FIG. 22B), and voltage (FIG. 22C). In general, a higher loading efficiency in the lumen of the EVs was observed when higher EV concentration over the cargo molecules and higher voltage were used.

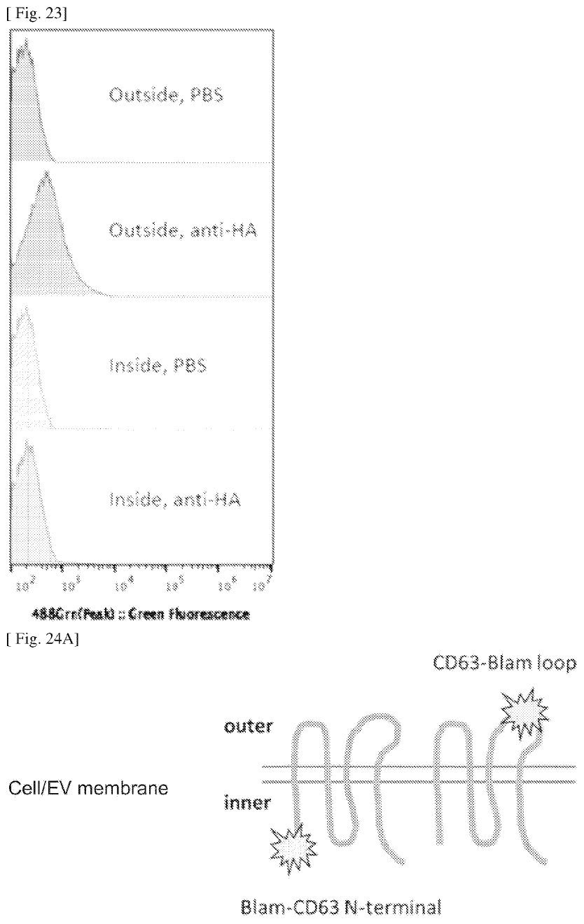

To determine whether the cargo molecules can be loaded outer portion or surface of the EVs, the CD46 Short scaffold construct of Example 4 was fused with an HA tag at the N-terminal (for outside) or C-terminal (inside) of the construct. Following overexpression in cells, the EVs were harvested, and the outside loading of the HA was measured by nFCM with anti-HA-488 (2350, CST) as shown in FIG. 23.

Generally, when the EVs arc uptaken by the target cells, the EVs translocate to the endosome where they fuse with the endosomal membrane to release the cargo. For the cargo molecules that are loaded in the lumen of the EVs, such as enzymes and nucleic acids, require endosomal escape in order to function in their target site within the cytoplasm. To study the release of the cargo molecules from the lumen of the EVs, beta-lactamase (BLAM) was used as a cargo molecule. CCF4-AM is a cell-permeable, non-fluorescent compound that can pass through the cell membrane due to its lipophilic nature. Once inside the cell, cellular esterases cleave the acetoxymethyl ester groups from CCF4-AM, resulting in the formation of the carboxyfluorescein molecule. The unique feature of CCF4 is that it can undergo a FRET-based color change upon interaction with beta-lactamase, an enzyme that hydrolyzes the beta-lactam ring present in antibiotics. In the intact state, CCF4 emits green fluorescence due to FRET between the donor fluorophore (carboxyfluorescein) and the acceptor fluorophore (coumarin). However, when beta-lactamase is active and present in the cellular environment, it cleaves the CCF4 molecule, resulting in the loss of FRET and a shift in fluorescence from green to blue. By introducing CCF4-AM into cells and monitoring the emission of fluorescence, the activity of beta-lactamase in the cytoplasm of the cell was assessed.

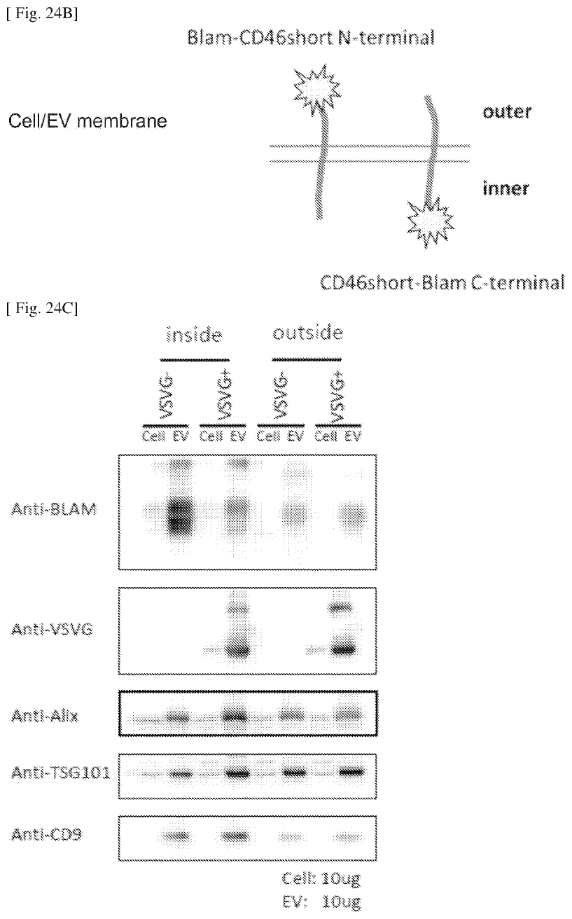

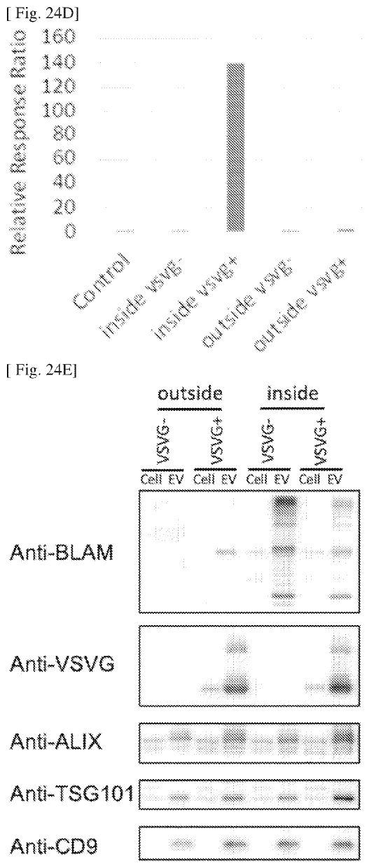

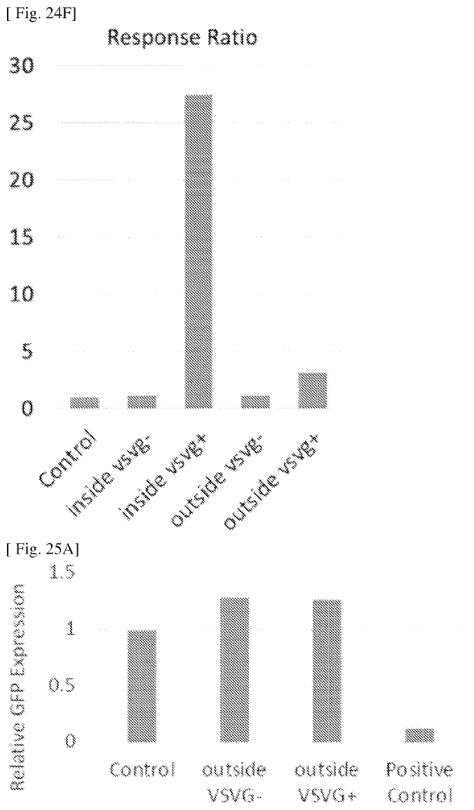

VSVG (Vesicular Stomatitis Virus Glycoprotein) is a viral protein, which can facilitate the escape of cargo from endosomes into the cytoplasm of the host cell. We fused BLAM to CD63 N-terminal (Inside) or extracellular loop (outside). In another sample, BLAM was fused to CD46-Short N-terminal (Inside) or C-terminal (outside). The EVs (carrying the fusion polypeptides either inside or outside of the vesicles) were harvested and taken up by the target 293T cells. The BLAM fusion polypeptides (i.e., BLAM-CD63-N-terminal, BLAM-CD63-extracellular loop, BLAM-CD46-Short-N-terminal, and CD46-Short-BLAM-C-terminal) were designed to express on the target cell or EVs membrane as illustrated in FIGS. 24A-B. As shown in FIGS. 24C-F, BLAM activity was observed only when the BLAM was placed inside of the EVs (not on the outer surface). Also, the results demonstrated that the overexpression of the cargo molecule release helper, VSVG, was required for the BLAM's cytoplasmic activity. The activity of BLAM was tested with LiveBLAzer™ FRET-B/G Loading Kit with CCF4-AM (K1096, ThermoFisher).

### Example 8: Extracellular Vesicles (EVs) Loaded with siRNA or shRNA

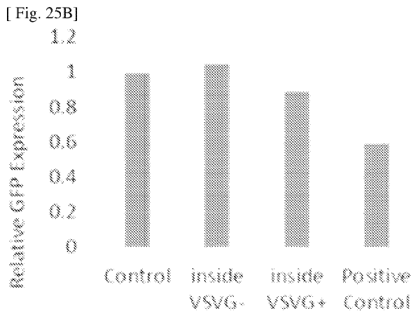

To determine whether the EVs can deliver the siRNA cargo molecules into the cells and provide sufficient knockdown efficiency, cholesterol modified siGFP labeled with Cy5 was loaded on the outer surface of the EVs (>90% loading efficiency determined by nFCM) and introduced to cells in the presence of absence of VSVG. Direct transfection with RNAiMAX was used as a positive control. No knockdown of the GFP was observed when the siRNA targeting GFP was loaded outside of the EVs. However, when the shGFP was loaded inside of the EVs in the presence or absence of VSVG in ExoBoost cells even with low loading efficiency of <10%, decrease in GFP expression level in cells was observed following shGFP-loaded EVs expression with VSVG (about 10% knockdown efficiency). The results are shown in FIGS. 25A-B.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### NUMBERED EMBODIMENTS OF THE DISCLOSURE

Other subject matter contemplated by the present disclosure is set out in the following numbered embodiments:
1. A method of enhancing extracellular vesicles (EVs) production, comprising: harvesting a plurality of EVs from a producer cell, wherein the producer cell is genetically engineered to overexpress at least one polypeptide, wherein the at least one polypeptide is linked to a glycosyl-phosphatidyl-inositol (GPI) group.
2. The method of embodiment 1, wherein the polypeptide is derived from any one of polypeptides in Table A.
3. The method of embodiment 1 or 2, wherein the polypeptide is derived from CD52, CD55, CD58, CD59, CD109, GPC1, GPC4, or GPC6.
4. The method of any one of embodiments 1-3, wherein the polypeptide is derived from CD59.
5. The method of any one of embodiments 1-3, wherein the polypeptide is derived from CD55.
6. The method of any one of embodiments 1-5, wherein the producer cell is genetically engineered by transfecting a recombinant vector system.
7. The method of embodiment 6, wherein the recombinant vector system comprises a nucleic acid sequence encoding the coding sequence of the polypeptide.
8. The method of embodiment 7, the recombinant vector system comprises an expression control sequence operably linked to the nucleic acid sequence.
9. The method of embodiment 8, wherein the nucleic acid sequence comprises at least one fluorescent marker.
10. The method of embodiment 8, wherein the expression control sequence is a promoter.
11. The method of embodiment 6, wherein the recombinant vector system comprises a selection marker.
12. The method of embodiment 11, wherein the selection marker is selected from the group consisting of neomycin resistance, puromycin resistance, hygromycin resistance, DHFR resistance, GPT resistance, zeocin resistance, G418 resistance, phleomycin resistance, blasticidin resistance, and histidinol resistance.
13. The method of embodiment 1, wherein the producer cell further comprises a release helper selected from the group consisting of Vesicular Stomatitis Virus Glycoprotein (VSVG), glycoprotein B (gB) of Herpes Simplex virus 1 (HSV-1), baculovirus fusion protein gp64, and gB from EpsteinBarr virus (EBV).
14. The method of any one of embodiments 1-13, wherein the producer cell is a genetically engineered stable cell line.
15. The method of any one of embodiments 1-14, wherein the plurality of EVs is harvested by dialysis or ultra-centrifugation.
16. The method of embodiment 15, wherein the plurality of EVs is harvested by ultra-centrifugation.
17. The method of any one of embodiments 1-16, wherein the concentration of the harvested EVs from the producer cell is at least 2-fold higher than those from a wild type cell.
18. The method of any one of embodiments 1-17, wherein the concentration of the harvested EVs from the producer cell is 2-fold to 250-fold higher than those from a wild type cell.
19. The method of any one of embodiments 1-18, wherein the producer cell is a mammalian cell.
20. The method of embodiment 19, wherein the producer cell is a HEK 293F cell, HEK 293T cell, mesenchymal stem cell (MSC), or any combination thereof.
21. The method of any one of embodiments 1-20, wherein the EVs is ectosomes, exosomes, microvesicles, apoptotic bodies, or any combination thereof.
22. The method of embodiment 21, wherein the EVs are exosomes.
23. The method of embodiment 1, wherein the cargo molecules comprise an active pharmaceutical ingredient (API).
24. The method of embodiment 23, wherein the API comprises small molecule therapeutics.
25. The method of embodiment 24, wherein the cargo molecule comprises a polypeptide, protein, lipid, nucleic acid, carbohydrate, metabolite, or any combinations thereof.
26. The method of embodiment 25, wherein the nucleic acid comprises DNA.
27. The method of embodiment 25, wherein the nucleic acid comprises peptide nucleic acids (PNAs).
28. The method of embodiment 25, wherein the nucleic acid comprises RNA.
29. The method of embodiment 28, wherein the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (lncRNA).
30. The method of embodiment 25, wherein the protein comprises an antibody or enzyme.
31. The method of embodiment 25, wherein the cargo molecule comprises an antisense oligonucleotide.
32. The method of embodiment 25, wherein the cargo molecule comprises a morpholino oligomer.
33. The method of embodiment 25, wherein the cargo molecule comprises one or more components of a gene editing system.
34. The method of embodiment 33, wherein the gene editing system is selected from the group consisting of CRISPR/Cas, zinc finger nuclease, transcription, and activator-like effector nuclease (TALEN).
35. The method of any one of embodiments 1-34, wherein the cargo molecule is located on the inner or outer surface of the plurality of EVs.
36. The method of embodiment 35, wherein the cargo molecule is a polypeptide fused with a polypeptide derived from CD46 or CD63.
37. The method of embodiment 35, wherein the cargo molecule is located on the inner surface of the plurality of EVs and wherein the cargo molecule has a higher efficacy in the presence of the release helper then without the presence of the release.
38. A method of making an extracellular vesicles (EVs) producing stable cell line, comprising:
a) transfecting an EV producer cell with an expression vector, wherein the expression vector comprises a nucleic acid sequence of one or more polypeptides and a selection marker, wherein the polypeptide is linked to a glycosyl-phosphatidyl-inositol (GPI) group;
b) screening and selecting the transfected cell; and
c) cultivating the selected cell.
39. The method of embodiment 38, wherein the polypeptide is derived from CD52, CD55, CD58, CD59, CD109, GPC1, GPC4, or GPC6.
40. The method of embodiment 39, wherein the polypeptide is derived from CD59.
41. The method of embodiment 39, wherein the polypeptide is derived from CD55.
42. The method of embodiment 38, the expression vector comprises an expression control sequence operably linked to the nucleic acid sequence.
43. The method of embodiment 42, the expression control sequence is a promoter.
44. The method of embodiment 42, wherein the nucleic acid sequence comprises at least one fluorescent marker.
45. The method of embodiment 38, wherein the selection marker is selected the group consisting of neomycin resistance, puromycin resistance, hygromycin resistance, DHFR resistance, GPT resistance, zeocin resistance, G418 resistance, phleomycin resistance, blasticidin resistance, and histidinol resistance.
46. The method of any one of embodiments 38-45, wherein the concentration of the harvested EVs from the producer cell is at least 2-fold higher than those from a wild type cell.
47. The method of any one of embodiments 38-46, wherein the concentration of the harvested EVs from the producer cell is 2-fold to 250-fold higher than those from a wild type cell.
48. The method of any one of embodiments 38-47, wherein the producer cell is a mammalian cell.
49. The method of embodiment 48, wherein the producer cell is a HEK 293F cell, HEK 293T cell, mesenchymal stem cell (MSC), or any combination thereof.
50. The method of any one of embodiments 38-49, wherein the EVs is ectosomes, exosomes, microvesicles, apoptotic bodies, or any combination thereof.
51. The method of embodiment 50, wherein the EVs are exosomes.
52. The method of any one of embodiments 38-51, wherein the EVs are loaded with cargo molecules.
53. The method of embodiment 52, wherein the cargo molecules comprise an active pharmaceutical ingredient (API).
54. The method of embodiment 53, wherein the API comprises small molecule therapeutics.
55. The method of embodiment 52, wherein the cargo molecule comprises a polypeptide, protein, lipid, nucleic acid, carbohydrate, metabolite, or any combinations thereof.
56. The method of embodiment 55, wherein the nucleic acid comprises DNA.
57. The method of embodiment 55, wherein the nucleic acid comprises peptide nucleic acids (PNAs).
58. The method of embodiment 55, wherein the nucleic acid comprises RNA.
59. The method of embodiment 58, wherein the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (lncRNA).
60. The method of embodiment 55, wherein the protein comprises an antibody or enzyme.
61. The method of embodiment 55, wherein the cargo molecule comprises antisense oligonucleotide.
62. The method of embodiment 55, wherein the cargo molecule comprises morpholino oligomer.
63. The method of embodiment 38, wherein the cargo molecule comprises one or more components of a gene editing system.
64. The method of embodiment 63, wherein the gene editing system is selected from the group consisting of CRISPR/Cas, zinc finger nuclease, transcription, and activator-like effector nuclease (TALEN).
65. A cell line manufactured according to any one of embodiments 38-64.
66. A kit for enhancing EVs production, comprising the producer cell of any one of embodiments 1-37 or the cell line of embodiment 65.
67. A composition comprising a plurality of EVs according to any one of embodiments 1-66.
68. The composition of embodiment 67, further comprising a pharmaceutically acceptable excipient.
69. A composition comprising an extracellular vesicles (EVs) producer cell, wherein the EV producer cell is genetically engineered to overexpress at least one or more polypeptides, wherein the polypeptide is linked to a glycosyl-phosphatidyl-inositol (GPI) group.
70. The composition of embodiment 69, wherein the polypeptide is derived from CD52, CD55, CD58, CD59, CD109, GPC1, GPC4, or GPC6.
71. The composition of embodiment 69 or 70, wherein the producer cell is genetically engineered by transfecting a recombinant vector system.
72. The composition of embodiment 71, wherein the recombinant vector system comprises a nucleic acid sequence encoding the coding sequence of the polypeptide.
73. The composition of embodiment 71, the recombinant vector system comprises an expression control sequence operably linked to the nucleic acid sequence.
74. The composition of embodiment 72, wherein the nucleic acid sequence comprises at least one fluorescent marker.
75. The composition of embodiment 73, the expression control sequence is a promoter.
76. The composition of embodiment 71, the recombinant vector system comprises a selection marker.
77. The composition of any one of embodiments 69-76, wherein the producer cell is a genetically engineered stable cell line.
78. The composition of embodiment 72, wherein the coding sequence of the polypeptide is selected from any one of sequences in Table 1.
79. The composition of embodiment 78, wherein the coding sequence of the polypeptide is mCherry-CD46 (Short), HA-CD46Short, or CD46Short-HA of Table 1.
80. The composition of embodiment 76, wherein the selection marker is selected the group consisting of neomycin resistance, puromycin resistance, hygromycin resistance, DHFR resistance, GPT resistance, zeocin resistance, G418 resistance, phleomycin resistance, blasticidin resistance, and histidinol resistance.
81. The composition of any one of embodiments 69-80, wherein the concentration of the harvested EVs from the producer cell is at least 2-fold higher than those from a wild type cell.
82. The composition of any one of embodiments 69-81, wherein the concentration of the harvested EVs from the producer cell is 2-fold to 250-fold higher than those from a wild type cell.
83. The composition of any one of embodiments 69-82, wherein the producer cell is a mammalian cell.
84. The composition of embodiment 83, wherein the producer cell is a HEK 293F cell, HEK 293T cell, mesenchymal stem cells (MSC) or any combination thereof.
85. The composition of any one of embodiments 69-84, wherein the EVs is ectosomes, exosomes, microvesicles, apoptotic bodies, or any combination thereof.
86. The composition of embodiment 85, wherein the EVs are exosomes.
87. The composition of any one of embodiments 69-86, wherein the EVs are loaded with cargo molecules.
88. The method of embodiment 87, wherein the cargo molecules comprise an active pharmaceutical ingredient (API).
89. The method of embodiment 88, wherein the API comprises small molecule therapeutics.
90. The method of embodiment 88, wherein the cargo molecule comprises a polypeptide, protein, lipid, nucleic acid, carbohydrate, metabolite, or any combinations thereof.
91. The method of embodiment 90, wherein the nucleic acid comprises DNA.
92. The method of embodiment 90, wherein the nucleic acid comprises peptide nucleic acids (PNAs).
93. The method of embodiment 90, wherein the nucleic acid comprises RNA.
94. The method of embodiment 93, wherein the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (lncRNA).
95. The method of embodiment 90, wherein the protein comprises an antibody or enzyme.
96. The method of embodiment 90, wherein the cargo molecule comprises antisense oligonucleotide.
97. The method of embodiment 90, wherein the cargo molecule comprises morpholino oligomer.
98. The method of embodiment 87, wherein the cargo molecule comprises one or more components of a gene editing system.
99. The method of embodiment 98, wherein the gene editing system is selected from the group consisting of CRISPR/Cas, zinc finger nuclease, transcription, and activator-like effector nuclease (TALEN).

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

1. A method of enhancing extracellular vesicles (EVs) production, comprising: harvesting a plurality of EVs from a producer cell, wherein the producer cell is genetically engineered to overexpress at least one polypeptide, and wherein the at least one polypeptide is linked to a glycosyl-phosphatidyl-inositol (GPI) group.

2. The method of claim 1, wherein the polypeptide is derived from any one of polypeptides in Table A.

3. The method of claim 1 or 2, wherein the polypeptide is derived from CD52, CD55, CD58, CD59, CD109, GPC1, GPC4, or GPC6.

4. The method of any one of claims 1-3, wherein the polypeptide is derived from CD59.

5. The method of any one of claims 1-3, wherein the polypeptide is derived from CD55.

6. The method of any one of claims 1-5, wherein the producer cell is genetically engineered by transfecting a recombinant vector system.

7. The method of claim 6, wherein the recombinant vector system comprises a nucleic acid sequence encoding the coding sequence of the polypeptide.

8. The method of claim 7, the recombinant vector system comprises an expression control sequence operably linked to the nucleic acid sequence.

9. The method of claim 8, wherein the nucleic acid sequence comprises at least one fluorescent marker.

10. The method of claim 8, wherein the expression control sequence is a promoter.

11. The method of claim 6, wherein the recombinant vector system comprises a selection marker.

12. The method of claim 11, wherein the selection marker is selected from the group consisting of neomycin resistance, puromycin resistance, hygromycin resistance, DHFR resistance, GPT resistance, zeocin resistance, G418 resistance, phleomycin resistance, blasticidin resistance, and histidinol resistance.

13. The method of claim 1, wherein the producer cell further comprises a release helper selected from the group consisting of Vesicular Stomatitis Virus Glycoprotein (VSVG), glycoprotein B (gB) of Herpes Simplex virus 1 (HSV-1), baculovirus fusion protein gp64, and gB from EpsteinBarr virus (EBV).

14. The method of any one of claims 1-13, wherein the producer cell is a genetically engineered stable cell line.

15. The method of any one of claims 1-14, wherein the plurality of EVs is harvested by dialysis or ultra-centrifugation.

16. The method of claim 15, wherein the plurality of EVs is harvested by ultra-centrifugation.

17. The method of any one of claims 1-16, wherein the concentration of the harvested EVs from the producer cell is at least 2-fold higher than those from a wild type cell.

18. The method of any one of claims 1-17, wherein the concentration of the harvested EVs from the producer cell is 2-fold to 250-fold higher than those from a wild type cell.

19. The method of any one of claims 1-18, wherein the producer cell is a mammalian cell.

20. The method of claim 19, wherein the producer cell is a HEK 293F cell, HEK 293T cell, mesenchymal stem cell (MSC), or any combination thereof.

21. The method of any one of claims 1-20, wherein the EVs is ectosomes, exosomes, microvesicles, apoptotic bodies, or any combination thereof.

22. The method of claim 21, wherein the EVs are exosomes.

23. The method of claim 1, wherein the EVs are loaded with a cargo molecule, wherein the cargo molecule comprises an active pharmaceutical ingredient (API).

24. The method of claim 23, wherein the API comprises small molecule therapeutics.

25. The method of claim 23, wherein the cargo molecule comprises a polypeptide, protein, lipid, nucleic acid, carbohydrate, metabolite, or any combinations thereof.

26. The method of claim 25, wherein the nucleic acid comprises DNA.

27. The method of claim 25, wherein the nucleic acid comprises peptide nucleic acids (PNAs).

28. The method of claim 25, wherein the nucleic acid comprises RNA.

29. The method of claim 28, wherein the RNA is selected from the group consisting of mRNA, small interfering RNA (siRNA), short hairpin RNAs (snoRNAs), antisense RNA, microRNA (mi-RNA), and long RNAs (snoRNAs), antisense RNA, microRNA (mi-RNA) and long non-coding RNA (lncRNA).

30. The method of claim 25, wherein the protein comprises an antibody or enzyme.

31. The method of claim 25, wherein the cargo molecule comprises an antisense oligonucleotide.

32. The method of claim 25, wherein the cargo molecule comprises a morpholino oligomer.

33. The method of claim 25, wherein the cargo molecule comprises one or more components of a gene editing system.

34. The method of claim 33, wherein the gene editing system is selected from the group consisting of CRISPR/Cas, zinc finger nuclease, transcription, and activator-like effector nuclease (TALEN).

35. The method of any one of claims 1-34, wherein the cargo molecule is located on the inner or outer surface of the plurality of EVs.

36. The method of claim 35, wherein the cargo molecule comprises a polypeptide fused with a polypeptide derived from CD46 or CD63.

37. The method of claim 35, wherein the cargo molecule is located on the inner surface of the plurality of EVs and wherein the cargo molecule has a higher efficacy in the presence of the release helper then without the presence of the release.

38. A method of making an extracellular vesicles (EVs) producing stable cell line, comprising:
a) transfecting an EV producer cell with an expression vector, wherein the expression vector comprises a nucleic acid sequence of one or more polypeptides and a selection marker, wherein the polypeptide is linked to a glycosyl-phosphatidyl-inositol (GPI) group;
b) screening and selecting the transfected cell; and
c) cultivating the selected cell.

39. The method of claim 38, wherein the polypeptide is derived from CD52, CD55, CD58, CD59, CD109, GPC1, GPC4, or GPC6.

40. The method of claim 39, wherein the polypeptide is derived from CD59.

41. The method of claim 39, wherein the polypeptide is derived from CD55.

42. The method of claim 38, the expression vector comprises an expression control sequence operably linked to the nucleic acid sequence.

43. The method of claim 42, the expression control sequence is a promoter.

44. The method of claim 42, wherein the nucleic acid sequence comprises at least one fluorescent marker.

45. The method of claim 38, wherein the selection marker is selected the group consisting of neomycin resistance, puromycin resistance, hygromycin resistance, DHFR resistance, GPT resistance, zeocin resistance, G418 resistance, phleomycin resistance, blasticidin resistance, and histidinol resistance.

46. The method of any one of claims 38-45, wherein the concentration of the harvested EVs from the producer cell is at least 2-fold higher than those from a wild type cell.

47. The method of any one of claims 38-46, wherein the concentration of the harvested EVs from the producer cell is 2-fold to 250-fold higher than those from a wild type cell.

48. The method of any one of claims 38-47, wherein the producer cell is a mammalian cell.

49. The method of claim 48, wherein the producer cell is a HEK 293F cell, HEK 293T cell, mesenchymal stem cell (MSC), or any combination thereof.

50. The method of any one of claims 38-49, wherein the EVs is ectosomes, exosomes, microvesicles, apoptotic bodies, or any combination thereof.

51. The method of claim 50, wherein the EVs are exosomes.

52. The method of any one of claims 38-51, wherein the EVs are loaded with cargo molecules.

53. The method of claim 52, wherein the cargo molecules comprise an active pharmaceutical ingredient (API).

54. The method of claim 53, wherein the API comprises small molecule therapeutics.

55. The method of claim 52, wherein the cargo molecule comprises a polypeptide, protein, lipid, nucleic acid, carbohydrate, metabolite, or any combinations thereof.

56. The method of claim 55, wherein the nucleic acid comprises DNA.

57. The method of claim 55, wherein the nucleic acid comprises peptide nucleic acids (PNAs).

58. The method of claim 55, wherein the nucleic acid comprises RNA.

59. The method of claim 58, wherein the RNA is selected from the group consisting of mRNA, small interfering RNA (siRNA), short hairpin RNAs (snoRNAs), antisense RNA, microRNA (mi-RNA), and long non-coding RNA (lncRNA).

60. The method of claim 55, wherein the protein comprises an antibody or enzyme.

61. The method of claim 55, wherein the cargo molecule comprises an antisense oligonucleotide.

62. The method of claim 55, wherein the cargo molecule comprises a morpholino oligomer.

63. The method of claim 38, wherein the cargo molecule comprises one or more components of a gene editing system.

64. The method of claim 63, wherein the gene editing system is selected from the group consisting of CRISPR/Cas, zinc finger nuclease, transcription, and activator-like effector nuclease (TALEN).

65. A cell line manufactured according to any one of claims 38-64.

66. A kit for enhancing EVs production, comprising the producer cell of any one of claims 1-37 or the cell line of claim 65.

67. A composition comprising a plurality of EVs according to any one of claims 1-66.

68. The composition of claim 67, further comprising a pharmaceutically acceptable excipient.

69. A composition comprising an extracellular vesicles (EVs) producer cell, wherein the EV producer cell is genetically engineered to overexpress at least one polypeptide, wherein the at least one polypeptide is linked to a glycosyl-phosphatidyl-inositol (GPI) group.

70. The composition of claim 69, wherein the polypeptide is derived from CD52, CD55, CD58, CD59, CD109, GPC1, GPC4, or GPC6.

71. The composition of claim 69 or 70, wherein the producer cell is genetically engineered by transfecting a recombinant vector system.

72. The composition of claim 71, wherein the recombinant vector system comprises a nucleic acid sequence encoding the coding sequence of the polypeptide.

73. The composition of claim 71, the recombinant vector system comprises an expression control sequence operably linked to the nucleic acid sequence.

74. The composition of claim 72, wherein the nucleic acid sequence comprises at least one fluorescent marker.

75. The composition of claim 73, the expression control sequence is a promoter.

76. The composition of claim 71, the recombinant vector system comprises a selection marker.

77. The composition of any one of claims 69-76, wherein the producer cell is a genetically engineered stable cell line.

78. The composition of claim 72, wherein the polypeptide is derived from any one of polypeptides in Table 1.

79. The composition of claim 78, wherein the polypeptide comprises a sequence of mCherry-CD46 (Short), HA-CD46Short, or CD46Short-HA of Table 1.

80. The composition of claim 76, wherein the selection marker is selected the group consisting of neomycin resistance, puromycin resistance, hygromycin resistance, DHFR resistance, GPT resistance, zeocin resistance, G418 resistance, phleomycin resistance, blasticidin resistance, and histidinol resistance.

81. The composition of any one of claims 69-80, wherein the concentration of the harvested EVs from the producer cell is at least 2-fold higher than those from a wild type cell.

82. The composition of any one of claims 69-81, wherein the concentration of the harvested EVs from the producer cell is 2-fold to 250-fold higher than those from a wild type cell.

83. The composition of any one of claims 69-82, wherein the producer cell is a mammalian cell.

84. The composition of claim 83, wherein the producer cell is a HEK 293F cell, HEK 293T cell, mesenchymal stem cells (MSC) or any combination thereof.

85. The composition of any one of claims 69-84, wherein the EVs is ectosomes, exosomes, microvesicles, apoptotic bodies, or any combination thereof.

86. The composition of claim 85, wherein the EVs are exosomes.

87. The composition of any one of claims 69-86, wherein the EVs are loaded with a cargo molecule.

88. The composition of claim 87, wherein the cargo molecule comprises an active pharmaceutical ingredient (API).

89. The composition of claim 88, wherein the API comprises small molecule therapeutics.

90. The composition of claim 88, wherein the cargo molecule comprises a polypeptide, protein, lipid, nucleic acid, carbohydrate, metabolite, or any combinations thereof.

91. The composition of claim 90, wherein the nucleic acid comprises DNA.

92. The composition of claim 90, wherein the nucleic acid comprises peptide nucleic acids (PNAs).

93. The composition of claim 90, wherein the nucleic acid comprises RNA.

94. The composition of claim 93, wherein the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (lncRNA).

95. The composition of claim 90, wherein the protein comprises an antibody or enzyme.

96. The composition of claim 90, wherein the cargo molecule comprises antisense oligonucleotide.

97. The composition of claim 90, wherein the cargo molecule comprises morpholino oligomer.

98. The composition of claim 87, wherein the cargo molecule comprises one or more components of a gene editing system.

99. The composition of claim 98, wherein the gene editing system is selected from the group consisting of CRISPR/Cas, zinc finger nuclease, transcription, and activator-like effector nuclease (TALEN).


SEQUENCE LISTING (22 sequences)

SEQ ID NO: 1 - protein, 381 aa, synthetic construct
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 2 - protein, 128 aa, synthetic construct
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 3 - protein, 392 aa, synthetic construct [sequence omitted]

SEQ ID NO: 4 - protein, 61 aa, synthetic construct
MKRFLFLLLTISLLVMVQIQTGLSGQNDTSQTSSPSASSNISGGIFLFFVANAIIHLFCFS

SEQ ID NO: 5 - protein, 397 aa, synthetic construct
MTVARPSVPAALPLLGELPRLLLLVLLCLPAVWGDYPYDVPDYAGSGSGSDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 6 - protein, 335 aa, synthetic construct
MTVARPSVPAALPLLGELPRLLLLVLLCLPAVWGDYPYDVPDYAGSGSGSSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 7 - protein, 271 aa, synthetic construct
MTVARPSVPAALPLLGELPRLLLLVLLCLPAVWGDYPYDVPDYAGSGSGSKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 8 - protein, 209 aa, synthetic construct
MTVARPSVPAALPLLGELPRLLLLVLLCLPAVWGDYPYDVPDYAGSGSGSIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 9 - protein, 146 aa, synthetic construct
MTVARPSVPAALPLLGELPRLLLLVLLCLPAVWGDYPYDVPDYAGSGSGSKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 10 - protein, 376 aa, synthetic construct
MTVARPSVPAALPLLGELPRLLLLVLLCLPAVWGDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 11 - protein, 626 aa, synthetic construct
MTVARPSVPAALPLLGELPRLLLLVLLCLPAVWGDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 12 - protein, 386 aa, synthetic construct
MEPPGRRECPFPSWRFPGLLLAAMVLLLYSFSDACVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 13 - protein, 637 aa, synthetic construct
MEPPGRRECPFPSWRFPGLLLAAMVLLLYSFSDACVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 14 - protein, 473 aa, synthetic construct
MVSKGEENNMAIIKEFMRFKVRMEGSVNGHEFEIEGEGEGRPYEGFQTAKLKVTKGGPLPFAWDILSPQFTYGSKAYVKHPADIPDYFKLSFPEGFKWERVMNFEDGGVVTVTQDSSLQDGEFIYKVKLRGTNFPSDGPVMQKKTMGWEASSERMYPEDGALKGEIKMRLKLKDGGHYTSEVKTTYKAKKPVQLPGAYIVGIKLDITSHNEDYTIVEQYERAEGRHSTGGMDELYKSGLRSRAQASPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 15 - protein, 483 aa, synthetic construct
MVSKGEENNMAIIKEFMRFKVRMEGSVNGHEFEIEGEGEGRPYEGFQTAKLKVTKGGPLPFAWDILSPQFTYGSKAYVKHPADIPDYFKLSFPEGFKWERVMNFEDGGVVTVTQDSSLQDGEFIYKVKLRGTNFPSDGPVMQKKTMGWEASSERMYPEDGALKGEIKMRLKLKDGGHYTSEVKTTYKAKKPVQLPGAYIVGIKLDITSHNEDYTIVEQYERAEGRHSTGGMDELYKSGLRSRAQASAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 16 - protein, 481 aa, synthetic construct
MVSKGEENNMAIIKEFMRFKVRMEGSVNGHEFEIEGEGEGRPYEGFQTAKLKVTKGGPLPFAWDILSPQFTYGSKAYVKHPADIPDYFKLSFPEGFKWERVMNFEDGGVVTVTQDSSLQDGEFIYKVKLRGTNFPSDGPVMQKKTMGWEASSERMYPEDGALKGEIKMRLKLKDGGHYTSEVKTTYKAKKPVQLPGAYIVGIKLDITSHNEDYTIVEQYERAEGRHSTGGMDELYKSGLRSRAQASGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 17 - protein, 321 aa, synthetic construct
MEPPGRRECPFPSWRFPGLLLAAMVLLLYSFSDACVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILASGLRSRAQASLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 18 - protein, 326 aa, synthetic construct
MEPPGRRECPFPSWRFPGLLLAAMVLLLYSFSDACSGLRSRAQASLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSLGGGGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 19 - protein, 156 aa, synthetic construct
MEPPGRRECPFPSWRFPGLLLAAMVLLLYSFSDACYPYDVPDYAGSGSGSLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 20 - protein, 156 aa, synthetic construct
MEPPGRRECPFPSWRFPGLLLAAMVLLLYSFSDACLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSLGSGSGSYPYDVPDYA

SEQ ID NO: 21 - protein, 410 aa, synthetic construct
MEPPGRRECPFPSWRFPGLLLAAMVLLLYSFSDACHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGSGSGSLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 22 - protein, 410 aa, synthetic construct
MEPPGRRECPFPSWRFPGLLLAAMVLLLYSFSDACLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSLGSGSGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW